(19) United States
(12) Patent Application Publication
(10) Pub. No.: US 2019/0015344 A1
(43) Pub. Date: Jan. 17, 2019

(54) FLOATING PHARMACEUTICAL DOSAGE FORM

(71) Applicant: GRÜNENTHAL GMBH, Aachen (DE)

(72) Inventors: Klaus WENING, Köln (DE); Carmen STOMBERG, Korschenbroich (DE); Marcel HAUPTS, Stolberg (DE)

(73) Assignee: GRÜNENTHAL GMBH, Aachen (DE)

(21) Appl. No.: 16/134,335

(22) Filed: Sep. 18, 2018

Related U.S. Application Data
(63) Continuation of application No. PCT/EP2017/056416, filed on Mar. 17, 2017.

(30) Foreign Application Priority Data
Mar. 18, 2016 (EP) 16161191.8

(51) Int. Cl. A61K 9/20; A61K 9/00; A61K 31/135

(52) U.S. Cl. CPC A61K 9/2095; A61K 9/0065; B33Y 80/00; A61K 9/204; A61K 9/2054; A61K 31/135

(57) ABSTRACT

The invention relates to a pharmaceutical dosage form having a density below the density of gastric fluid, wherein the dosage form comprises a pharmacologically active ingredient and a cavity. The invention also relates to a process for the preparation of said dosage form comprising a three-dimensional printing step.

This application is a continuation of International Patent Application No. PCT/EP2017/056416, filed Mar. 17, 2017, which claims priority of European Patent Application No. 16161191.8, filed Mar. 18, 2016, the entire contents of which patent applications are hereby incorporated herein by reference.

The invention relates to a pharmaceutical dosage form having a density below the density of gastric fluid, wherein the dosage form comprises a pharmacologically active ingredient and a cavity. The invention also relates to a process for the preparation of said dosage form comprising a three-dimensional printing step.

The design of oral controlled drug delivery system should be primarily aimed at achieving more predictable and increased bioavailability of drugs. A major constraint in oral controlled release drug delivery is that not all drug candidates are absorbed uniformly throughout the gastro intestinal tract. Some drugs are absorbed in a particular absorption window. After crossing absorption window, the released drug gets wasted with negligible or no absorption. Thus the time available for drug absorption drastically decreases. Also most of the drugs are sparingly soluble or insoluble in gastric fluids. In such drugs, dissolution and bioavailability are directly dependent on time available for solubilization and thus gastric retention time. Thus, bioavailability of such drugs can be increased by prolonging the gastric retention time. Gastro-retentive drug delivery system increases the gastric residence time of drug providing a better opportunity for increased solubilization and bioavailability. Also it can help in controlling the drug release rate by using various swellable polymer systems. Various approaches of gastro-retention are available such as bioadhesive dosage forms, floating drug delivery by density based approach or effervescent system etc. (see Mundade et al., Digest Journal of Nanomaterials and Biostructures, 2014, 9(3), 1077-1084).

Gastric retentive drug delivery provides a promising technology exhibiting an extended gastric residence and a drug release independent of patient related variables. It is usually useful in improving local gastric treatment as well as overcoming drug-related problems, i.e. drugs having narrow absorption window, short half-life or low intestinal solubility.

U.S. Pat. No. 3,976,764 discloses a solid therapeutic preparation for gastric diseases, remaining in stomach for a long period of time and gradually releasing pharmacologically active ingredient contained therein into gastric fluid while remaining in stomach, prepared by impregnation of the active ingredient together with other suitable additives into a body of empty globular shell or a granular lump in small size of the material having high buoyancy. These preparations have a large total volume, as the rely upon conventional capsules which are provided with an outer coating material containing the drug thereby further increasing the total volume. The larger the volume the poorer the patient compliance. Further, the mechanical strength of these dosage forms is comparatively poor, as the body of globular shell (e.g. capsule) has a poor mechanical strength as well. Thus, it is difficult to achieve permanent adherence of the outer material to the body during shipping and storage. Furthermore, this reference does not relate to dosage forms which have been manufactured by three dimensional printing technology.

U.S. Pat. No. 5,626,876 relates to a floatable, oral, therapeutic system, in which a lengthening of the gastrointestinal residence time of medicaments and a controlled delivery thereof are achieved by systems, which are specifically lighter than the gastric fluid, float on the latter and can only with difficulty reach the lower-lying pylorus, said system using at least one structural element with cavities or voids, such as foams or hollow bodies. This reference does not relate to dosage forms which have been manufactured by three dimensional printing technology.

US 2003/0021846 relates to oral dosage forms comprising a) one or more active ingredients, b) a formulated mixture of polyvinyl acetate and polyvinylpyrrolidone, c) where appropriate, other excipients customary for producing the dosage form, wherein they float on gastric fluid and display delayed release of active ingredient, and to the use and production thereof.

US 2006/0003003 relates to a sustained release formulation with gastric retention properties comprising tedisamil or a pharmaceutically acceptable salt thereof and the use of this formulation in the prevention and treatment of atrial fibrillation, atrial flutter and cardiac ischemia.

WO 2010/143052 provides a pharmaceutical composition comprising pregabalin that is useful for once daily oral dosing. Disclosed is a gastro-retentive tablet comprising pregabalin and one or more water insoluble component wherein water insoluble component preferably comprises a combination of ethylcellulose and hydrogenated castor oil.

WO 2016/038356 discloses 3D-printing technology to produce solid dosage forms, such as pharmaceutical tablets and relates to in situ (e.g. within a pharmacy) 3D-printing of personalized medicines tailored to a patient's needs. This reference further relates to dosage forms having different release profiles, which were determined in a two-step process, i.e. the tablets were tested for two hours in a simulated gastric fluid at pH 1.2 followed by 16 hours exposure to phosphate buffer at pH 6.8. This reference is not drawn to dosage forms which remain in a floating state in the stomach and provide release of a pharmacologically active ingredient in the stomach for a longer period than two hours.

Goyanes et al., “Fused-filament 3D printing for fabrication of tablets”, Int. J. Pharmaceutics, Vol. 476, No. 1-2, September 2014, p. 88.92; relate to the use of fused-filament 3D printing to fabricate individual tables and disclose that the release profiles of the printed tablets were dependent on the infill percentage used to print the tablet. This reference does not aim at achieving gastric-retention and release of the active ingredient into the gastric fluid. On the contrary, in vitro release is determined at pH 6.8, i.e. under intestinal conditions.

B. K. Almutairy et al., 2016 annual meeting of the American Association of Pharmaceutical Scientists, Colo., Denver, USA, poster 30W1230, disclose a floating drug delivery system prepared via hot-melt extrusion technology coupled with pressurized CO₂ for a thermo-labile API. The pressurized CO₂ foams the material such that the resultant foam contains micropores that are filled with CO₂.

Buoyancy is considered one of the most promising approaches for gastro-retention of dosage forms. Floating drug delivery systems have a bulk density lower than gastric fluids and thus remain buoyant in the stomach causing an increase in gastric residence time. The buoyancy of these systems is attained by the aid of substances responsible to generate the low density. Various agents with different mechanisms were adopted either gas-generating agents, air entrapping swellable polymers, inherent low density substances, porous excipients, hollow/porous particles inducing preparation techniques or sublimating agents (see Ishak, J Pharm Pharm Sci 2015, 18(1), 77-100).

The gastro-retentive dosage forms of the prior art are not satisfactory in every respect and there is a demand for improved gastro-retentive dosage forms. Such improved gastro-retentive dosage forms should be easy to manufacture, provide reliable and reproducible drug release and should principally be useful for a large variety of different drugs and combinations thereof.

It is therefore an object of the invention to provide pharmaceutical dosage forms having advantages over the pharmaceutical dosage forms of the prior art.

This object has been achieved by the subject-matter of the patent claims.

It has been surprisingly found that three-dimensional printing technology, preferably fused deposition modeling, makes available pharmaceutical dosage forms comprising one or more cavities providing sufficient buoyancy such that the pharmaceutical dosage forms float in gastric fluid. In contrast to conventional manufacturing processes such as direct compression, granulation or extrusion, three-dimensional printing technology allows for precisely predetermining the individual volume of the one or more cavities that cause buoyancy (i.e., gastric retention) of the pharmaceutical dosage form.

Pharmaceutical dosage forms comprising cavities have not been available by conventional manufacturing processes, as the cavities form sites of mechanical weakness that would fracture under the conditions of conventional manufacturing processes, especially under high pressure. Furthermore, precise positioning of cavities within pharmaceutical dosage forms by conventional means is extremely difficult, whereas it has been unexpectedly found that this can be easily achieved by means of three-dimensional printing.

Three-dimensional printing technology allows for precisely predetermining individual size, individual shape, individual position and the like of each cavity within the pharmaceutical dosage form. By adjusting these parameters, the properties of the pharmaceutical dosage form (e.g. release of pharmacologically active ingredient, density, buoyancy and the like) can be predetermined not only for a state of the pharmaceutical dosage form prior to its administration to a patient, but also for a state after administration, i.e. when the dosage form has already been subjected to erosion in gastric fluid for a certain period of time to a certain extent. As erosion and other mechanisms taking place during exposure to gastric fluid cause dissolution of material forming the outer surface of the pharmaceutical dosage form (only this material is in direct contact with gastric fluid), the resulting shrinkage of the overall volume of the pharmaceutical dosage form over time may be predicted.

Conventional pharmaceutical dosage forms are made of a material having a density which is typically greater than that of gastric fluid (otherwise conventional dosage forms would float). When the pharmaceutical dosage form is made e.g. from a foamed material in order to reduce its density, a plurality of micropores is homogeneously distributed over the dosage form. The size of the individual micropores (gas bubbles) in the foam is very difficult to control. Their position and size distribution is random and hence also very difficult to control if not impossible. When such dosage forms are exposed to gastric fluid, the overall density of the dosage form will not substantially change over time, as micropores located in the surrounding spheres will continuously erode and be filled with gastric fluid, respectively.

In contrast, when precisely predetermining individual size, individual shape, individual position and the like of each cavity within the pharmaceutical dosage form, a change of density, particularly a decrease of density, may be achieved over time. For example, when one or two cavities of predetermined macroscopic size and shape are located in the core section of a pharmaceutical dosage form which is surrounded by a mass of material having a greater density than gastric fluid, the overall dosage form may be floating due to the cavities. After exposure to gastric fluid for a certain period of time, material will dissolve from the surroundings without any influence on size and shape of the one or two cavities. Thus, the resultant overall density of the eroded dosage form will be lower than that of the dosage form prior to administration.

Further, three-dimensional printing technology also allows for manufacturing pharmaceutical dosage forms from different materials, i.e. from different pharmaceutical compositions, which in the pharmaceutical dosage form are arranged at different locations thus forming separate subunits of the dosage form. Compared to conventional techniques, e.g. coating techniques, said two different materials may be positioned in rather complex arrangements thus having precise shapes which may be e.g. interlocked with one another, but do not need to be merely layered on one another. Said separate subunits may serve different purposes. While one subunit may serve e.g. the purpose of releasing the pharmacologically active ingredient while the dosage form remains in the floating state in the gastric fluid, another subunit may serve e.g. the purpose of providing and maintaining the floating state.

Thus, for example, an enteric material may surround and thus protect the one or more cavities such that they cannot be filled with (penetrated by) gastric fluid and maintain buoyancy of the dosage form over time, and a non-enteric material containing the pharmacologically active ingredient may be exposed to gastric fluid thereby allowing for erosion and release during buoyancy. Such use and purpose of an enteric material is unconventional. It has been surprisingly found that enteric materials are not only useful for protecting the active pharmaceutical ingredient from being released under acidic conditions like in conventional enteric dosage forms, but are also useful for protecting subunits of the pharmaceutical dosage form that provide floating (buoyant) properties, i.e. gastric retentive properties, from being filled with gastric fluid. In the first case (conventional), the purpose of the enteric material is to avoid release of the pharmacologically active ingredient in the gastric fluid. In the latter case (inventive), the purpose is just the opposite, namely to maintain gastric retention thereby releasing as much as possible of the pharmacologically active ingredient in the gastric fluid.

A first aspect of the invention relates to a pharmaceutical dosage form having a density below the density of gastric fluid, wherein the dosage form comprises a pharmacologically active ingredient and a cavity. Preferably, the dosage form is gastric-retentive and provides release of the pharmacologically active ingredient in the stomach. As the pharmaceutical dosage form has a density below the density of gastric fluid, it spontaneously floats in gastric fluid after oral intake. Preferably, the dosage form floats in gastric fluid for a sufficient period of time such that the majority of the pharmacologically active ingredient is released during its floating state in the stomach after oral intake. Preferably, the dosage form comprises at least one cavity having a volume of at least 0.1 μl, preferably alt least 0.5 μl, more preferably at least 1.0 μl.

A skilled person knows how to distinguish gastric-retentive dosage forms from non-gastric-retentive dosage forms. For the purpose of the specification, a gastric-retentive dosage form retains in the stomach for a sufficient time interval against all the physiological barriers (e.g. gastric motility, gastric emptying, and the like), releases pharmacologically active ingredient into the gastric fluid, and finally is excreted by the body, optionally metabolized. Preferably, a gastric-retentive dosage form is retained in the stomach during the housekeeper wave (phase III of the interdigestive myoelectric cycle or migrating myoelectric complex).

The dosage form according to the invention has been manufactured by three-dimensional printing technology, preferably by fused-deposition modeling. Methods to distinguish such dosage forms from other dosage forms that have been manufactured by conventional techniques such as direct compression, extrusion, wet granulation, dry granulation, and the like are known to the skilled person and include but are not limited to microscopy and electron microscopy.

Density of gastric fluid typically varies between 1.001 to 1.010 g/mL. As the density of the pharmaceutical dosage form according to the invention is below that of gastric fluid, the pharmaceutical dosage form is capable of floating (swimming) in gastric fluid. The density of the pharmaceutical dosage form is its overall density, i.e. total weight of the pharmaceutical dosage form (including the cavity) divided by the total outer volume of the pharmaceutical dosage form. The total outer volume is defined by the outer silhouette of the dosage form. Typically, the cavity is filled with gas and thus, as such has a lower density than the remainder of the material forming the dosage form. Therefore, the overall density of the dosage form results from the combination of the relatively low density of the cavity filling (gas) with the relatively high density of the remainder of the material forming the dosage form including material encasing the cavity. The pharmaceutical dosage form according to the invention may also be referred to as “floating dosage form” or “buoyant dosage form”. In consequence of these properties, the pharmaceutical dosage form according to the invention has gastro-retentive properties and is particularly useful for pharmacologically active ingredients that are advantageously released in the stomach but not in the intestine.

Preferably, the pharmaceutical dosage form according to the invention has a density of at most 1.000 g/mL, or at most 0.999 g/mL, or at most 0.998 g/mL, or at most 0.997 g/mL, or at most 0.996 g/mL, or at most 0.995 g/mL, or at most 0.994 g/mL, or at most 0.993 g/mL, or at most 0.992 g/mL, or at most 0.991 g/mL; more preferably at most 0.990 g/mL, or at most 0.989 g/mL, or at most 0.988 g/mL, or at most 0.987 g/mL, or at most 0.986 g/mL, or at most 0.985 g/mL, or at most 0.984 g/mL, or at most 0.983 g/mL, or at most 0.982 g/mL, or at most 0.981 g/mL; still more preferably at most 0.980 g/mL, or at most 0.979 g/mL, or at most 0.978 g/mL, or at most 0.977 g/mL, or at most 0.976 g/mL, or at most 0.975 g/mL, or at most 0.974 g/mL, or at most 0.973 g/mL, or at most 0.972 g/mL, or at most 0.971 g/mL; yet more preferably at most 0.970 g/mL, or at most 0.969 g/mL, or at most 0.968 g/mL, or at most 0.967 g/mL, or at most 0.966 g/mL, or at most 0.965 g/mL, or at most 0.964 g/mL, or at most 0.963 g/mL, or at most 0.962 g/mL, or at most 0.961 g/mL; even more preferably at most 0.060 g/mL, or at most 0.959 g/mL, or at most 0.958 g/mL, or at most 0.957 g/mL, or at most 0.956 g/mL, or at most 0.955 g/mL, or at most 0.954 g/mL, or at most 0.953 g/mL, or at most 0.952 g/mL, or at most 0.951 g/mL; most preferably at most 0.950 g/mL, or at most 0.949 g/mL, or at most 0.948 g/mL, or at most 0.947 g/mL, or at most 0.946 g/mL, or at most 0.945 g/mL, or at most 0.944 g/mL, or at most 0.943 g/mL, or at most 0.942 g/mL, or at most 0.941 g/mL; and in particular at most 0.940 g/mL, or at most 0.939 g/mL, or at most 0.938 g/mL, or at most 0.937 g/mL, or at most 0.936 g/mL, or at most 0.935 g/mL, or at most 0.934 g/mL, or at most 0.933 g/mL, or at most 0.932 g/mL, or at most 0.931 g/mL.

In a preferred embodiment, the density of the pharmaceutical dosage form according to the invention is within the range of 0.900±0.100 g/mL, more preferably 0.900±0.090 g/mL, still more preferably 0.900±0.080 g/mL, yet more preferably 0.900±0.070 g/mL, even more preferably 0.900±0.060 g/mL, most preferably 0.900±0.050 g/mL, and in particular 0.900±0.040 g/mL.

In another preferred embodiment, the density of the pharmaceutical dosage form according to the invention is within the range of 0.850±0.100 g/mL, more preferably 0.850±0.090 g/mL, still more preferably 0.850±0.080 g/mL, yet more preferably 0.850±0.070 g/mL, even more preferably 0.850±0.060 g/mL, most preferably 0.850±0.050 g/mL, and in particular 0.850±0.040 g/mL.

In still another preferred embodiment, the density of the pharmaceutical dosage form according to the invention is within the range of 0.800±0.100 g/mL, more preferably 0.800±0.090 g/mL, still more preferably 0.800±0.080 g/mL, yet more preferably 0.800±0.070 g/mL, even more preferably 0.800±0.060 g/mL, most preferably 0.800±0.050 g/mL, and in particular 0.800±0.040 g/mL.

In another preferred embodiment, the density of the pharmaceutical dosage form according to the invention is within the range of 0.750±0.100 g/mL, more preferably 0.750±0.090 g/mL, still more preferably 0.750±0.080 g/mL, yet more preferably 0.750±0.070 g/mL, even more preferably 0.750±0.060 g/mL, most preferably 0.750±0.050 g/mL, and in particular 0.750±0.040 g/mL.

In another preferred embodiment, the density of the pharmaceutical dosage form according to the invention is within the range of 0.700±0.100 g/mL, more preferably 0.700±0.090 g/mL, still more preferably 0.700±0.080 g/mL, yet more preferably 0.700±0.070 g/mL, even more preferably 0.700±0.060 g/mL, most preferably 0.700±0.050 g/mL, and in particular 0.700±0.040 g/mL.

In a preferred embodiment, the pharmaceutical dosage form according to the invention is characterized by a density gradient, i.e. by an inhomogeneous distribution of material of different density. It has been surprisingly found that by three-dimensional printing technology this can be easily achieved. The density gradient may extend along one of the directions of extension of the pharmaceutical dosage form.

The pharmacologically active ingredient that is contained in the pharmaceutical dosage form is not particularly limited. The pharmaceutical dosage form may contain a single pharmacologically active ingredient or a combination of two or more pharmacologically active ingredients or a combination of two or more derivatives of one and the same pharmacologically active ingredients such as different salts.

Preferably, the pharmacologically active ingredient is advantageously released in the stomach but not in the intestine. In a preferred embodiment, the pharmacologically active ingredient is not stable at pH values of 6.8 and above so that it should be absorbed under the acidic conditions in the stomach. In another preferred embodiment, bioavailability of the pharmacologically active ingredient under the acidic conditions is higher than at pH values of 6.8 and above so that absorption in the stomach is desirable. In still another preferred embodiment, the pharmacologically active ingredient has fewer or no side effects when being absorbed under the acidic conditions of the stomach, compared to absorption in the intestine.

Preferably, the dosage form provides release of the pharmacologically active ingredient into the gastric fluid, i.e. in the stomach. Under physiological conditions, preferably at least 40 wt.-%, more preferably at least 50 wt.-%, still more preferably at least 60 wt.-%, yet more preferably at least 70 wt.-%, even more preferably at least 80 wt.-%, most preferably at least 90 wt.-%, and in particular about the total quantity of the pharmacologically active ingredient that was originally contained in the dosage form are released into the gastric fluid (i.e. within the stomach).

Pharmacologically active ingredients satisfying any one or a combination of the above requirements are known to the skilled person. Examples include but are not limited to sitagliptin, metformin, gabapentin, ciprofloxacin, misoprostol, L-dopa, benserazide, diazepam, and the like.

In a preferred embodiment, the pharmacologically active ingredient is for the treatment of diseases or disorders of the nervous system [ATC code N]; more preferably an analgesic [ATC code N02], or an antiepileptic [ATC code N03], or a psychoanaleptic [ATC code N06]; most preferably an opioid [ATC code N02A], an antiepileptic [ATC code N03A], or a psychostimulant [ATC code N06B]. Other pharmacologically active ingredients include but are not limited to gastric acid secretion inhibitors, gastric acid neutralizers or anti-pepsin inhibitors (e.g. propantheline bromide or benactyzine hydrochloride).

Preferably, the dosage form has a total volume of at most 800 μl, or at most 775 μl, or at most 750 μl, or at most 725 μl, or at most 700 μl, or at most 675 μl, or at most 650 μl, or at most 625 μl, or at most 600 μl, or at most 575 μl, or at most 550 μl, or at most 525 μl, or at most 500 μl, or at most 475 μl, or at most 450 μl, or at most 425 μl, or at most 400 μl, or at most 390 μl, or at most 380 μl, or at most 370 μl, or at most 360 μl, or at most 350 μl, or at most 340 μl, or at most 330 μl, or at most 320 μl, or at most 310 μl, or at most 300 μl, or at most 290 μl, or at most 280 μl, or at most 270 μl, or at most 260 μl, or at most 250 μl, or at most 240 μl, or at most 230 μl, or at most 220 μl, or at most 210 μl, or at most 200 μl, or at most 190 μl, or at most 180 μl, or at most 170 μl, or at most 160 μl, or at most 150 μl, or at most 140 μl, or at most 130 μl, or at most 120 μl, or at most 110 μl, or at most 100 μl, or at most 90 μl, or at most 80 μl, or at most 70 μl, or at most 60 μl, or at most 50 μl, or at most 40 μl, or at most 30 μl, or at most 20 μl, or at most 10 μl, or at most 5 μl.

The pharmaceutical dosage form according to the invention comprises a cavity. The cavity is contained in the inside of the pharmaceutical dosage form and preferably not visible from the outside with the naked eye (see FIG. 1). The cavity is embedded within the pharmaceutical dosage form and has no opening to the surroundings (i.e. is closed). Thus, the pharmaceutical dosage form according to the invention preferably may be regarded as a hollow body, wherein the cavity forms a hollow space (void). The cavity is of macroscopic size, i.e. microscopic interspaces between powder particles that remain after compression of a powdery material are not to be regarded as cavity in the meaning of the invention.

Preferably, the pharmaceutical dosage form according to the invention comprises at least one cavity having a volume of at least 0.1 μl, or at least 0.5 μl, or at least 1.0 μl, or at least 1.5 μl, or at least 2.0 μl, or at least 2.5 μl, or at least 3.0 μl, or at least 3.5 μl, or at least 4.0 μl, or at least 4.5 μl, or at least 5.0 μl; more preferably at least 5.5 μl, or at least 6.0 μl, or at least 6.5 μl, or at least 7.0 μl, or at least 7.5 μl, or at least 8.0 μl, or at least 8.5 μl, or at least 9.0 μl, or at least 9.5 μl, or at least 10 μl; still more preferably at least 11 μl, or at least 12 μl, or at least 13 μl, or at least 14 μl, or at least 15 μl, or at least 16 μl, or at least 17 μl, or at least 18 μl, or at least 19 μl, or at least 20 μl; yet more preferably at least 21 μl, or at least 22 μl, or at least 23 μl, or at least 24 μl, or at least 25 μl, or at least 26 μl, or at least 27 μl, or at least 28 μl, or at least 29 μl, or at least 30 μl; even more preferably at least 41 μl, or at least 42 μl, or at least 43 μl, or at least 44 μl, or at least 45 μl, or at least 46 μl, or at least 47 μl, or at least 48 μl, or at least 49 μl, or at least 50 μl; most preferably at least 51 μl, or at least 52 μl, or at least 53 μl, or at least 54 μl, or at least 55 μl, or at least 56 μl, or at least 57 μl, or at least 58 μl, or at least 59 μl, or at least 60 μl; and most preferably at least 61 μl, or at least 62 μl, or at least 63 μl, or at least 64 μl, or at least 65 μl, or at least 66 μl, or at least 67 μl, or at least 68 μl, or at least 69 μl, or at least 70 μl.

Preferably, the pharmaceutical dosage form according to the invention comprises at least one cavity having a volume of at most 500 μl, or at most 490 μl, or at most 480 μl, or at most 470 μl, or at most 460 μl, or at most 450 μl, or at most 440 μl, or at most 440 μl, or at most 420 μl, or at most 410 μl, or at most 400 μl, or at most 390 μl, or at most 380 μl, or at most 370 μl, or at most 360 μl, or at most 350 μl, or at most 340 μl, or at most 330 μl, or at most 320 μl, or at most 310 μl, or at most 300 μl, or at most 290 μl, or at most 280 μl, or at most 270 μl, or at most 260 μl, or at most 250 μl, or at most 240 μl, or at most 230 μl, or at most 220 μl, or at most 210 μl, or at most 200 μl, or at most 190 μl, or at most 180 μl, or at most 170 μl, or at most 160 μl, or at most 150 μl, or at most 140 μl, or at most 130 μl, or at most 120 μl, or at most 110 μl, or at most 100 μl, or at most 99 μl, or at most 98 μl, or at most 97 μl, or at most 96 μl, or at most 95 μl, or at most 94 μl, or at most 93 μl, or at most 92 μl, or at most 91 μl; more preferably at most 90 μl, or at most 89 μl, or at most 88 μl, or at most 87 μl, or at most 86 μl, or at most 85 μl, or at most 84 μl, or at most 83 μl, or at most 82 μl, or at most 81 μl; still more preferably at most 80 μl, or at most 89 μl, or at most 88 μl, or at most 87 μl, or at most 86 μl, or at most 85 μl, or at most 84 μl, or at most 83 μl, or at most 82 μl, or at most 81 μl; yet more preferably at most 79 μl, or at most 78 μl, or at most 77 μl, or at most 76 μl, or at most 75 μl, or at most 74 μl, or at most 73 μl, or at most 72 μl, or at most 71 μl, or at most 70 μl; even more preferably at most 69 μl, or at most 68 μl, or at most 67 μl, or at most 66 μl, or at most 65 μl, or at most 64 μl, or at most 63 μl, or at most 62 μl, or at most 61 μl, or at most 60 μl; and most preferably at most 59 μl, or at most 58 μl, or at most 57 μl, or at most 56 μl, or at most 55 μl, or at most 54 μl, or at most 53 μl, or at most 52 μl, or at most 51 μl, or at most 50 μl, or at most 49 μl, or at most 48 μl, or at most 47 μl, or at most 46 μl, or at most 45 μl, or at most 44 μl, or at most 43 μl, or at most 42 μl, or at most 41 μl.

Preferably, the at least one cavity has a volume in the range of 5.0±4.5 μl, or of 5.0±4.0 μl, or of 5.0±3.5 μl, or of 5.0±3.0 μl, or of 5.0±2.5 μl, or of 5.0±2.0 μl, or of 5.0±1.5 μl, or of 5.0±1.0 μl; more preferably in the range of 10.0±9.0 μl, or of 10.0±8.0 μl, or of 10.0±7.0 μl, or of 10.0±6.0 μl, or of 10.0±5.0 μl, or of 10.0±4.0 μl, or of 10.0±3.0 μl, or of 10.0±2.0 μl, or of 10.0±1.0 μl; or of 20.0±16.0 μl, or of 20.0±14.0 μl, or of 20.0±12.0 μl, or of 20.0±10.0 μl, or of 20.0±9.0 μl, or of 20.0±8.0 μl, or of 20.0±7.0 μl, or of 20.0±6.0 μl, or of 20.0±5.0 μl, or of 20.0±4.0 μl, or of 20.0±3.0 μl, or of 20.0±2.0 μl, or of 20.0±1.0 μl; or of 30.0±26.0 μl, or of 30.0±24.0 μl, or of 30.0±22.0 μl, or of 30.0±20.0 μl, or of 30.0±18.0 μl, or of 30.0±16.0 μl, or of 30.0±14.0 μl, or of 30.0±12.0 μl, or of 30.0±10.0 μl, or of 30.0±9.0 μl, or of 30.0±8.0 μl, or of 30.0±7.0 μl, or of 30.0±6.0 μl, or of 30.0±5.0 μl, or of 30.0±4.0 μl, or of 30.0±3.0 μl, or of 30.0±2.0 μl, or of 30.0±1.0 μl; or of 40.0±15.0 μl, or of 40.0±10.0 μl, or of 40.0±5.0 μl, or of 50.0±15.0 μl, or of 50.0±10.0 μl, or of 50.0±5.0 μl, or of 60.0±15.0 μl, or of 60.0±10.0 μl, or of 60.0±5.0 μl, or of 70.0±15.0 μl, or of 70.0±10.0 μl, or of 70.0±5.0 μl, or of 80.0±15.0 μl, or of 80.0±10.0 μl, or of 80.0±5.0 μl, or of 90.0±15.0 μl, or of 90.0±10.0 μl, or of 90.0±5.0 μl, or of 100.0±50.0 μl, or of 100.0±40.0 μl, or of 100.0±30.0 μl, or of 100.0±20.0 μl, or of 100.0±10.0 μl, or of 150.0±50.0 μl, or of 150.0±40.0 μl, or of 150.0±30.0 μl, or of 150.0±20.0 μl, or of 150.0±10.0 μl, or of 200.0±50.0 μl or of 200.0±40.0 μl, or of 200.0±30.0 μl, or of 200.0±20.0 μl, or of 200.0±10.0 μl, or of 250.0±50.0 μl, or of 250.0±40.0 μl, or of 250.0±30.0 μl, or of 250.0±20.0 μl, or of 250.0±10.0 μl, or of 300.0±50.0 μl or of 300.0±40.0 μl, or of 300.0±30.0 μl or of 300.0±20.0 μl, or of 300.0±10.0 μl, or of 350.0±50.0 μl, or of 350.0±40.0 μl, or of 350.0±30.0 μl, or of 350.0±20.0 μl, or of 350.0±10.0 μl, or of 400.0±50.0 μl or of 400.0±40.0 μl, or of 400.0±30.0 μl or of 400.0±20.0 μl, or of 400.0±10.0 μl, or of 450.0±50.0 μl, or of 450.0±40.0 μl, or of 450.0±30.0 μl, or of 450.0±20.0 μl, or of 450.0±10.0 μl, or of 500.0±50.0 μl or of 500.0±40.0 μl, or of 500.0±30.0 μl or of 500.0±20.0 μl, or of 500.0±10.0 μl, or of 550.0±50.0 μl, or of 550.0±40.0 μl, or of 550.0±30.0 μl, or of 550.0±20.0 μl, or of 550.0±10.0 μl.

In a preferred embodiment, the at least one cavity has a volume which is at least 1.0 vol.-%, or at least 2.0 vol.-%, or at least 3.0 vol.-%, or at least 4.0 vol.-%, or at least 5.0 vol.-%; more preferably at least 6.0 vol.-%, or at least 7.0 vol.-%, or at least 8.0 vol.-%, or at least 9.0 vol.-%, or at least 10.0 vol.-%; still more preferably at least 11.0 vol.-%, or at least 12.0 vol.-%, or at least 13.0 vol.-%, or at least 14.0 vol.-%, or at least 15.0 vol.-%; yet more preferably at least 16.0 vol.-%, or at least 17.0 vol.-%, or at least 18.0 vol.-%, or at least 19.0 vol.-%, or at least 20.0 vol.-%; even more preferably at least 21.0 vol.-%, or at least 22.0 vol.-%, or at least 23.0 vol.-%, or at least 24.0 vol.-%, or at least 25.0 vol.-%; most preferably at least 26.0 vol.-%, or at least 27.0 vol.-%, or at least 28.0 vol.-%, or at least 29.0 vol.-%, or at least 30.0 vol.-%; and in particular at least 31.0 vol.-%, or at least 32.0 vol.-%, or at least 33.0 vol.-%, or at least 34.0 vol.-%, or at least 35.0 vol.-%, or at least 36.0 vol.-%, or at least 37.0 vol.-%, or at least 38.0 vol.-%, or at least 39.0 vol.-%, or at least 40.0 vol.-%, or at least 41.0 vol.-%, or at least 42.0 vol.-%, or at least 43.0 vol.-%, or at least 44.0 vol.-%, or at least 45.0 vol.-%, in each case of the total volume of the dosage form.

In a preferred embodiment, the at least one cavity has a volume which is at most 50.0 vol.-%, or at most 49.0 vol.-%, or at most 48.0 vol.-%, or at most 47.0 vol.-%, or at most 46.0 vol.-%, or at most 45.0 vol.-%, or at most 44.0 vol.-%, or at most 43.0 vol.-%, at most 42.0 vol.-%, or at most 41.0 vol.-%, or at most 40.0 vol.-%; more preferably at most 39.0 vol.-%, or at most 38.0 vol.-%, or at most 37.0 vol.-%; or at most 36.0 vol.-%, or at most 35.0 vol.-%, or at most 34.0 vol.-%, or at most 33.0 vol.-%; or at most 32.0 vol.-%, or at most 31.0 vol.-%, or at most 30.0 vol.-%; still more preferably at most 29.0 vol.-%, or at most 28.0 vol.-%, or at most 27.0 vol.-%; or at most 26.0 vol.-%, or at most 25.0 vol.-%, or at most 24.0 vol.-%, or at most 23.0 vol.-%; or at most 22.0 vol.-%, or at most 31.0 vol.-%, or at most 20.0 vol.-%; even more preferably at most 19.0 vol.-%, or at most 18.0 vol.-%, or at most 17.0 vol.-%; or at most 16.0 vol.-%, or at most 15.0 vol.-%, or at most 14.0 vol.-%, or at most 13.0 vol.-%; or at most 12.0 vol.-%, or at most 11.0 vol.-%, or at most 10.0 vol.-%; most preferably at most 9.0 vol.-%, or at most 8.0 vol.-%, or at most 7.0 vol.-%; or at most 6.0 vol.-%, or at most 5.0 vol.-%, or at most 4.0 vol.-%, in each case of the total volume of the dosage form.

Preferably, the at least one cavity has a volume in the range of 5.0±4.5 vol.-%, or of 5.0±4.0 vol.-%, or of 5.0±3.5 vol.-%, or of 5.0±3.0 vol.-%, or of 5.0±2.5 vol.-%, or of 5.0±2.0 vol.-%, or of 5.0±1.5 vol.-%, or of 5.0±1.0 vol.-%; more preferably in the range of 10.0±9.0 vol.-%, or of 10.0±8.0 vol.-%, or of 10.0±7.0 vol.-%, or of 10.0±6.0 vol.-%, or of 10.0±5.0 vol.-%, or of 10.0±4.0 vol.-%, or of 10.0±3.0 vol.-%, or of 10.0±2.0 vol.-%, or of 10.0±1.0 vol.-%; still more preferably in the range of 15.0±9.0 vol.-%, or of 15.0±8.0 vol.-%, or of 15.0±7.0 vol.-%, or of 15.0±6.0 vol.-%, or of 15.0±5.0 vol.-%, or of 15.0±4.0 vol.-%, or of 15.0±3.0 vol.-%, or of 15.0±2.0 vol.-%, or of 15.0±1.0 vol.-%, or of 20.0±16.0 vol.-%, or of 20.0±14.0 vol.-%, or of 20.0±12.0 vol.-%, or of 20.0±10.0 vol.-%, or of 20.0±9.0 vol.-%, or of 20.0±8.0 vol.-%, or of 20.0±7.0 vol.-%, or of 20.0±6.0 vol.-%, or of 20.0±5.0 vol.-%, or of 20.0±4.0 vol.-%, or of 20.0±3.0 vol.-%, or of 20.0±2.0 vol.-%, or of 20.0±1.0 vol.-%; yet more preferably in the range of 25.0±16.0 vol.-%, or of 25.0±14.0 vol.-%, or of 25.0±12.0 vol.-%, or of 25.0±10.0 vol.-%, or of 25.0±9.0 vol.-%, or of 25.0±8.0 vol.-%, or of 25.0±7.0 vol.-%, or of 25.0±6.0 vol.-%, or of 25.0±5.0 vol.-%, or of 25.0±4.0 vol.-%, or of 25.0±3.0 vol.-%, or of 25.0±2.0 vol.-%, or of 25.0±1.0 vol.-%, or of 30.0±26.0 vol.-%, or of 30.0±24.0 vol.-%, or of 30.0±22.0 vol.-%, or of 30.0±20.0 vol.-%, or of 30.0±18.0 vol.-%, or of 30.0±16.0 vol.-%, or of 30.0±14.0 vol.-%, or of 30.0±12.0 vol.-%, or of 30.0±10.0 vol.-%, or of 30.0±9.0 vol.-%, or of 30.0±8.0 vol.-%, or of 30.0±7.0 vol.-%, or of 30.0±6.0 vol.-%, or of 30.0±5.0 vol.-%, or of 30.0±4.0 vol.-%, or of 30.0±3.0 vol.-%, or of 30.0±2.0 vol.-%, or of 30.0±1.0 vol.-%; even more preferably in the range of 35.0±20.0 vol.-%, or of 35.0±18.0 vol.-%, or of 35.0±16.0 vol.-%, or of 35.0±14.0 vol.-%, or of 35.0±12.0 vol.-%, or of 35.0±10.0 vol.-%, or of 35.0±9.0 vol.-%, or of 35.0±8.0 vol.-%, or of 35.0±7.0 vol.-%, or of 35.0±6.0 vol.-%, or of 35.0±5.0 vol.-%, or of 35.0±4.0 vol.-%, or of 35.0±3.0 vol.-%, or of 35.0±2.0 vol.-%, or of 35.0±1.0 vol.-%; most preferably in the range of 40.0±16.0 vol.-%, or of 40.0±14.0 vol.-%, or of 40.0±12.0 vol.-%, or of 40.0±10.0 vol.-%, or of 40.0±9.0 vol.-%, or of 40.0±8.0 vol.-%, or of 40.0±7.0 vol.-%, or of 40.0±6.0 vol.-%, or of 40.0±5.0 vol.-%, or of 40.0±4.0 vol.-%, or of 40.0±3.0 vol.-%, or of 40.0±2.0 vol.-%, or of 40.0±1.0 vol.-%; or of 45.0±10.0 vol.-%, or of 45.0±9.0 vol.-%, or of 45.0±8.0 vol.-%, or of 45.0±7.0 vol.-%, or of 45.0±6.0 vol.-%, or of 45.0±5.0 vol.-%, or of 45.0±4.0 vol.-%, or of 45.0±3.0 vol.-%, or of 45.0±2.0 vol.-%, or of 45.0±1.0 vol.-%; and in particular in the range of 50.0±10.0 vol.-%, or of 50.0±9.0 vol.-%, or of 50.0±8.0 vol.-%, or of 50.0±7.0 vol.-%, or of 50.0±6.0 vol.-%, or of 50.0±5.0 vol.-%, or of 50.0±4.0 vol.-%, or of 50.0±3.0 vol.-%, or of 50.0±2.0 vol.-%, or of 50.0±1.0 vol.-%, or of 55.0±5.0 vol.-%, or of 55.0±4.0 vol.-%, or of 55.0±3.0 vol.-%, or of 55.0±2.0 vol.-%, or of 55.0±1.0 vol.-%, in each case of the total volume of the dosage form.

In a preferred embodiment, the pharmaceutical dosage form according to the invention comprises a single cavity (i.e. not more than one cavity).

In another preferred embodiment, the pharmaceutical dosage form according to the invention comprises more than one cavity, e.g. at least 2 cavities, at least 3 cavities, at least 4 cavities, at least 5 cavities, at least 6 cavities or more. The at least 2 cavities may substantially have similar or the same volume or different volumes. Preferably, the at least 2 cavities have about the same volume.

In another preferred embodiment, the pharmaceutical dosage form according to the invention comprises at most 6 cavities, at most 5 cavities, at most 4 cavities, at most 3 cavities, or at most 2 cavities.

Preferably, the pharmaceutical dosage form comprises 1, 2, 3, 4, 5 or 6 cavities.

In a preferred embodiment, the pharmaceutical dosage form comprises web layers above one another thereby forming cavities. The web is preferably formed by three dimensionally printing thereby leaving indentations where no material is printed. According to this embodiment, the web layers are preferably surrounded by a closed shell such that the web structure is not visible from the outside with the naked eye. Preferably, upon three-dimensional printing, a first layer is printed that assumes the shape of a web and a second layer is printed on top of the first layer that also assumes the shape of a web, whereas preferably the web structure of the first layer is displaced relative to the web structure of the second layer such that the indentations in the web structure of the first layer are preferably closed by the printed material of the second web layer thereby forming cavities that preferably do not extend from the first layer to the second layer, but are positioned within the first layer and the second layer, respectively. A skilled person recognizes that on top of the second layer, a third layer may be printed that also assumes the shape of a web, and so on.

In a preferred embodiment, the pharmaceutical dosage form comprises in its inside at least one layer that assumes the shape of a web, preferably at least two layers that assume the shape of a web, more preferably at least three layers that assume the shape of a web, and still more preferably at least four layers that assume the shape of a web.

Preferably, the total volume of all cavities that are comprised in the pharmaceutical dosage form according to the invention is at least 0.5 μl, or at least 1.0 μl, or at least 1.5 μl, or at least 2.0 μl, or at least 2.5 μl, or at least 3.0 μl, or at least 3.5 μl, or at least 4.0 μl, or at least 4.5 μl, or at least 5.0 μl; more preferably at least 5.5 μl, or at least 6.0 μl, or at least 6.5 μl, or at least 7.0 μl, or at least 7.5 μl, or at least 8.0 μl, or at least 8.5 μl, or at least 9.0 μl, or at least 9.5 μl, or at least 10 μl; still more preferably at least 11 μl, or at least 12 μl, or at least 13 μl, or at least 14 μl, or at least 15 μl, or at least 16 μl, or at least 17 μl, or at least 18 μl, or at least 19 μl, or at least 20 μl; yet more preferably at least 21 μl, or at least 22 μl, or at least 23 μl, or at least 24 μl, or at least 25 μl, or at least 26 μl, or at least 27 μl, or at least 28 μl, or at least 29 μl, or at least 30 μl; even more preferably at least 41 μl, or at least 42 μl, or at least 43 μl, or at least 44 μl, or at least 45 μl, or at least 46 μl, or at least 47 μl, or at least 48 μl, or at least 49 μl, or at least 50 μl; most preferably at least 51 μl, or at least 52 μl, or at least 53 μl, or at least 54 μl, or at least 55 μl, or at least 56 μl, or at least 57 μl, or at least 58 μl, or at least 59 μl, or at least 60 μl; and most preferably at least 61 μl, or at least 62 μl, or at least 63 μl, or at least 64 μl, or at least 65 μl, or at least 66 μl, or at least 67 μl, or at least 68 μl, or at least 69 μl, or at least 70 μl.

Preferably, the total volume of all cavities that are comprised in the pharmaceutical dosage form according to the invention is at most 500 μl, or at most 490 μl, or at most 480 μl, or at most 470 μl, or at most 460 μl, or at most 450 μl, or at most 440 μl, or at most 440 μl, or at most 420 μl, or at most 410 μl, or at most 400 μl, or at most 390 μl, or at most 380 μl, or at most 370 μl, or at most 360 μl, or at most 350 μl, or at most 340 μl, or at most 330 μl, or at most 320 μl, or at most 310 μl, or at most 300 μl, or at most 290 μl, or at most 280 μl, or at most 270 μl, or at most 260 μl, or at most 250 μl, or at most 240 μl, or at most 230 μl, or at most 220 μl, or at most 210 μl, or at most 200 μl, or at most 190 μl, or at most 180 μl, or at most 170 μl, or at most 160 μl, or at most 150 μl, or at most 140 μl, or at most 130 μl, or at most 120 μl, or at most 110 μl, or at most 100 μl, or at most 99 μl, or at most 98 μl, or at most 97 μl, or at most 96 μl, or at most 95 μl, or at most 94 μl, or at most 93 μl, or at most 92 μl, or at most 91 μl; more preferably at most 90 μl, or at most 89 μl, or at most 88 μl, or at most 87 μl, or at most 86 μl, or at most 85 μl, or at most 84 μl, or at most 83 μl, or at most 82 μl, or at most 81 μl; still more preferably at most 80 μl, or at most 89 μl, or at most 88 μl, or at most 87 μl, or at most 86 μl, or at most 85 μl, or at most 84 μl, or at most 83 μl, or at most 82 μl, or at most 81 μl; yet more preferably at most 79 μl, or at most 78 μl, or at most 77 μl, or at most 76 μl, or at most 75 μl, or at most 74 μl, or at most 73 μl, or at most 72 μl, or at most 71 μl, or at most 70 μl; even more preferably at most 69 μl, or at most 68 μl, or at most 67 μl, or at most 66 μl, or at most 65 μl, or at most 64 μl, or at most 63 μl, or at most 62 μl, or at most 61 μl, or at most 60 μl; and most preferably at most 59 μl, or at most 58 μl, or at most 57 μl, or at most 56 μl, or at most 55 μl, or at most 54 μl, or at most 53 μl, or at most 52 μl, or at most 51 μl, or at most 50 μl, or at most 49 μl, or at most 48 μl, or at most 47 μl, or at most 46 μl, or at most 45 μl, or at most 44 μl, or at most 43 μl, or at most 42 μl, or at most 41 μl.

Preferably, the total volume of all cavities that are comprised in the pharmaceutical dosage form according to the invention is in the range of 5.0±4.5 μl, or of 5.0±4.0 μl, or of 5.0±3.5 μl, or of 5.0±3.0 μl, or of 5.0±2.5 μl, or of 5.0±2.0 μl, or of 5.0±1.5 μl, or of 5.0±1.0 μl; more preferably in the range of 10.0±9.0 μl, or of 10.0±8.0 μl, or of 10.0±7.0 μl, or of 10.0±6.0 μl, or of 10.0±5.0 μl, or of 10.0±4.0 μl, or of 10.0±3.0 μl, or of 10.0±2.0 μl, or of 10.0±1.0 μl; or of 20.0±16.0 μl, or of 20.0±14.0 μl, or of 20.0±12.0 μl, or of 20.0±10.0 μl, or of 20.0±9.0 μl, or of 20.0±8.0 μl, or of 20.0±7.0 μl, or of 20.0±6.0 μl, or of 20.0±5.0 μl, or of 20.0±4.0 μl, or of 20.0±3.0 μl, or of 20.0±2.0 μl, or of 20.0±1.0 μl; or of 30.0±26.0 μl, or of 30.0±24.0 μl, or of 30.0±22.0 μl, or of 30.0±20.0 μl, or of 30.0±18.0 μl, or of 30.0±16.0 μl, or of 30.0±14.0 μl, or of 30.0±12.0 μl, or of 30.0±10.0 μl, or of 30.0±9.0 μl, or of 30.0±8.0 μl, or of 30.0±7.0 μl, or of 30.0±6.0 μl, or of 30.0±5.0 μl, or of 30.0±4.0 μl, or of 30.0±3.0 μl, or of 30.0±2.0 μl, or of 30.0±1.0 μl; or of 40.0±15.0 μl, or of 40.0±10.0 μl, or of 40.0±5.0 μl, or of 50.0±15.0 μl, or of 50.0±10.0 μl, or of 50.0±5.0 μl, or of 60.0±15.0 μl, or of 60.0±10.0 μl, or of 60.0±5.0 μl, or of 70.0±15.0 μl, or of 70.0±10.0 μl, or of 70.0±5.0 μl, or of 80.0±15.0 μl, or of 80.0±10.0 μl, or of 80.0±5.0 μl, or of 90.0±15.0 μl, or of 90.0±10.0 μl, or of 90.0±5.0 μl, or of 100.0±50.0 μl, or of 100.0±40.0 μl, or of 100.0±30.0 μl, or of 100.0±20.0 μl, or of 100.0±10.0 μl, or of 150.0±50.0 μl, or of 150.0±40.0 μl, or of 150.0±30.0 μl, or of 150.0±20.0 μl, or of 150.0±10.0 μl, or of 200.0±50.0 μl or of 200.0±40.0 μl, or of 200.0±30.0 μl, or of 200.0±20.0 μl, or of 200.0±10.0 μl, or of 250.0±50.0 μl, or of 250.0±40.0 μl, or of 250.0±30.0 μl, or of 250.0±20.0 μl, or of 250.0±10.0 μl, or of 300.0±50.0 μl or of 300.0±40.0 μl, or of 300.0±30.0 μl or of 300.0±20.0 μl, or of 300.0±10.0 μl, or of 350.0±50.0 μl, or of 350.0±40.0 μl, or of 350.0±30.0 μl, or of 350.0±20.0 μl, or of 350.0±10.0 μl, or of 400.0±50.0 μl or of 400.0±40.0 μl, or of 400.0±30.0 μl or of 400.0±20.0 μl, or of 400.0±10.0 μl, or of 450.0±50.0 μl, or of 450.0±40.0 μl, or of 450.0±30.0 μl, or of 450.0±20.0 μl, or of 450.0±10.0 μl, or of 500.0±50.0 μl or of 500.0±40.0 μl, or of 500.0±30.0 μl or of 500.0±20.0 μl, or of 500.0±10.0 μl, or of 550.0±50.0 μl, or of 550.0±40.0 μl, or of 550.0±30.0 μl, or of 550.0±20.0 μl, or of 550.0±10.0 μl.

In a preferred embodiment, the total volume of all cavities that are comprised in the pharmaceutical dosage form according to the invention is at least 1.0 vol.-%, or at least 2.0 vol.-%, or at least 3.0 vol.-%, or at least 4.0 vol.-%, or at least 5.0 vol.-%; more preferably at least 6.0 vol.-%, or at least 7.0 vol.-%, or at least 8.0 vol.-%, or at least 9.0 vol.-%, or at least 10.0 vol.-%; still more preferably at least 11.0 vol.-%, or at least 12.0 vol.-%, or at least 13.0 vol.-%, or at least 14.0 vol.-%, or at least 15.0 vol.-%; yet more preferably at least 16.0 vol.-%, or at least 17.0 vol.-%, or at least 18.0 vol.-%, or at least 19.0 vol.-%, or at least 20.0 vol.-%; even more preferably at least 21.0 vol.-%, or at least 22.0 vol.-%, or at least 23.0 vol.-%, or at least 24.0 vol.-%, or at least 25.0 vol.-%; most preferably at least 26.0 vol.-%, or at least 27.0 vol.-%, or at least 28.0 vol.-%, or at least 29.0 vol.-%, or at least 30.0 vol.-%; and in particular at least 31.0 vol.-%, or at least 32.0 vol.-%, or at least 33.0 vol.-%, or at least 34.0 vol.-%, or at least 35.0 vol.-%, or at least 36.0 vol.-%, or at least 37.0 vol.-%, or at least 38.0 vol.-%, or at least 39.0 vol.-%, or at least 40.0 vol.-%, or at least 41.0 vol.-%, or at least 42.0 vol.-%, or at least 43.0 vol.-%, or at least 44.0 vol.-%, or at least 45.0 vol.-%, in each case of the total volume of the dosage form.

In a preferred embodiment, the total volume of all cavities that are comprised in the pharmaceutical dosage form according to the invention is at most 50.0 vol.-%, or at most 49.0 vol.-%, or at most 48.0 vol.-%, or at most 47.0 vol.-%, or at most 46.0 vol.-%, or at most 45.0 vol.-%, or at most 44.0 vol.-%, or at most 43.0 vol.-%, at most 42.0 vol.-%, or at most 41.0 vol.-%, or at most 40.0 vol.-%; more preferably at most 39.0 vol.-%, or at most 38.0 vol.-%, or at most 37.0 vol.-%; or at most 36.0 vol.-%, or at most 35.0 vol.-%, or at most 34.0 vol.-%, or at most 33.0 vol.-%; or at most 32.0 vol.-%, or at most 31.0 vol.-%, or at most 30.0 vol.-%; still more preferably at most 29.0 vol.-%, or at most 28.0 vol.-%, or at most 27.0 vol.-%; or at most 26.0 vol.-%, or at most 25.0 vol.-%, or at most 24.0 vol.-%, or at most 23.0 vol.-%; or at most 22.0 vol.-%, or at most 31.0 vol.-%, or at most 20.0 vol.-%; even more preferably at most 19.0 vol.-%, or at most 18.0 vol.-%, or at most 17.0 vol.-%; or at most 16.0 vol.-%, or at most 15.0 vol.-%, or at most 14.0 vol.-%, or at most 13.0 vol.-%; or at most 12.0 vol.-%, or at most 11.0 vol.-%, or at most 10.0 vol.-%; most preferably at most 9.0 vol.-%, or at most 8.0 vol.-%, or at most 7.0 vol.-%; or at most 6.0 vol.-%, or at most 5.0 vol.-%, or at most 4.0 vol.-%, in each case of the total volume of the dosage form.

Preferably, the total volume of all cavities that are comprised in the pharmaceutical dosage form according to the invention is in the range of 5.0±4.5 vol.-%, or of 5.0±4.0 vol.-%, or of 5.0±3.5 vol.-%, or of 5.0±3.0 vol.-%, or of 5.0±2.5 vol.-%, or of 5.0±2.0 vol.-%, or of 5.0±1.5 vol.-%, or of 5.0±1.0 vol.-%; more preferably in the range of 10.0±9.0 vol.-%, or of 10.0±8.0 vol.-%, or of 10.0±7.0 vol.-%, or of 10.0±6.0 vol.-%, or of 10.0±5.0 vol.-%, or of 10.0±4.0 vol.-%, or of 10.0±3.0 vol.-%, or of 10.0±2.0 vol.-%, or of 10.0±1.0 vol.-%; still more preferably in the range of 15.0±9.0 vol.-%, or of 15.0±8.0 vol.-%, or of 15.0±7.0 vol.-%, or of 15.0±6.0 vol.-%, or of 15.0±5.0 vol.-%, or of 15.0±4.0 vol.-%, or of 15.0±3.0 vol.-%, or of 15.0±2.0 vol.-%, or of 15.0±1.0 vol.-%, or of 20.0±16.0 vol.-%, or of 20.0±14.0 vol.-%, or of 20.0±12.0 vol.-%, or of 20.0±10.0 vol.-%, or of 20.0±9.0 vol.-%, or of 20.0±8.0 vol.-%, or of 20.0±7.0 vol.-%, or of 20.0±6.0 vol.-%, or of 20.0±5.0 vol.-%, or of 20.0±4.0 vol.-%, or of 20.0±3.0 vol.-%, or of 20.0±2.0 vol.-%, or of 20.0±1.0 vol.-%; yet more preferably in the range of 25.0±16.0 vol.-%, or of 25.0±14.0 vol.-%, or of 25.0±12.0 vol.-%, or of 25.0±10.0 vol.-%, or of 25.0±9.0 vol.-%, or of 25.0±8.0 vol.-%, or of 25.0±7.0 vol.-%, or of 25.0±6.0 vol.-%, or of 25.0±5.0 vol.-%, or of 25.0±4.0 vol.-%, or of 25.0±3.0 vol.-%, or of 25.0±2.0 vol.-%, or of 25.0±1.0 vol.-%, or of 30.0±26.0 vol.-%, or of 30.0±24.0 vol.-%, or of 30.0±22.0 vol.-%, or of 30.0±20.0 vol.-%, or of 30.0±18.0 vol.-%, or of 30.0±16.0 vol.-%, or of 30.0±14.0 vol.-%, or of 30.0±12.0 vol.-%, or of 30.0±10.0 vol.-%, or of 30.0±9.0 vol.-%, or of 30.0±8.0 vol.-%, or of 30.0±7.0 vol.-%, or of 30.0±6.0 vol.-%, or of 30.0±5.0 vol.-%, or of 30.0±4.0 vol.-%, or of 30.0±3.0 vol.-%, or of 30.0±2.0 vol.-%, or of 30.0±1.0 vol.-%; even more preferably in the range of 35.0±20.0 vol.-%, or of 35.0±18.0 vol.-%, or of 35.0±16.0 vol.-%, or of 35.0±14.0 vol.-%, or of 35.0±12.0 vol.-%, or of 35.0±10.0 vol.-%, or of 35.0±9.0 vol.-%, or of 35.0±8.0 vol.-%, or of 35.0±7.0 vol.-%, or of 35.0±6.0 vol.-%, or of 35.0±5.0 vol.-%, or of 35.0±4.0 vol.-%, or of 35.0±3.0 vol.-%, or of 35.0±2.0 vol.-%, or of 35.0±1.0 vol.-%; most preferably in the range of 40.0±16.0 vol.-%, or of 40.0±14.0 vol.-%, or of 40.0±12.0 vol.-%, or of 40.0±10.0 vol.-%, or of 40.0±9.0 vol.-%, or of 40.0±8.0 vol.-%, or of 40.0±7.0 vol.-%, or of 40.0±6.0 vol.-%, or of 40.0±5.0 vol.-%, or of 40.0±4.0 vol.-%, or of 40.0±3.0 vol.-%, or of 40.0±2.0 vol.-%, or of 40.0±1.0 vol.-%; or of 45.0±10.0 vol.-%, or of 45.0±9.0 vol.-%, or of 45.0±8.0 vol.-%, or of 45.0±7.0 vol.-%, or of 45.0±6.0 vol.-%, or of 45.0±5.0 vol.-%, or of 45.0±4.0 vol.-%, or of 45.0±3.0 vol.-%, or of 45.0±2.0 vol.-%, or of 45.0±1.0 vol.-%; and in particular in the range of 50.0±10.0 vol.-%, or of 50.0±9.0 vol.-%, or of 50.0±8.0 vol.-%, or of 50.0±7.0 vol.-%, or of 50.0±6.0 vol.-%, or of 50.0±5.0 vol.-%, or of 50.0±4.0 vol.-%, or of 50.0±3.0 vol.-%, or of 50.0±2.0 vol.-%, or of 50.0±1.0 vol.-%, or of 55.0±5.0 vol.-%, or of 55.0±4.0 vol.-%, or of 55.0±3.0 vol.-%, or of 55.0±2.0 vol.-%, or of 55.0±1.0 vol.-%, in each case of the total volume of the dosage form.

The total weight of the pharmaceutical dosage form according to the invention is not particularly limited. Preferably, the pharmaceutical dosage form according to the invention has a total weight within the range of 100±50 mg, or 200±100 mg, or 300±150 mg, or 400±200 mg, or 500±250 mg, or 600±300 mg, or 700±350 mg, or 800±400 mg, or 900±450 mg, or 1000±500 mg.

Preferably, the pharmaceutical dosage form according to the invention is monolithic. Preferably, the pharmaceutical dosage form according to the invention is not multiparticulate, e.g. dose not contain a multitude of hollow microspheres or a porous material. Preferably, the pharmaceutical dosage form according to the invention is a tablet.

Preferably, the pharmaceutical dosage form according to the invention, although comprising at least one cavity, has a certain degree of mechanical strength. Preferably, the pharmaceutical dosage form according to the invention has a breaking strength of at least 25 N, more preferably at least 30 N, still more preferably at least 35 N, yet more preferably at least 40 N, even more preferably at least 45 N, most preferably at least 50 N, and in particular at least 55 N. Preferably, the breaking strength is determined in accordance with Ph. Eur. (2.9.8 Resistance to crushing of tablets). In preferred embodiments, the breaking strength of the pharmaceutical dosage form according to the invention is at least 60 N, or at least 70 N, or at least 80 N, or at least 90 N, or at least 100 N, or at least 110 N, or at least 120 N, or at least 130 N, or at least 140 N, or at least 150 N, or at least 160 N, or at least 170 N, or at least 180 N, or at least 190 N, or at least 200 N.

Preferably, the pharmaceutical dosage form according to the invention is made of a single or of two different materials but does not comprise any subunits which are made of a further material. In this regard “material” refers to any homogenous composition of matter that is useful for the manufacture of pharmaceutical dosage forms by three-dimensional printing technology. Such composition may contain the pharmacologically active ingredient and one or more excipients.

The outer shape of the pharmaceutical dosage form according to the invention is not particularly limited. Preferably, the pharmaceutical dosage form according to the invention is
round or oblong and/or
flat or biconvex.

Typically, the pharmaceutical dosage form can be swallowed by a patient without causing any harm. Preferably, the outer surface of the pharmaceutical dosage form does substantially not comprise and concave portions.

Preferably, the single cavity or the at least 2 cavities of the pharmaceutical dosage form are filled with gas. The gas essentially provides sufficient buoyancy such that the pharmaceutical dosage form is capable of floating in gastric fluid. The gas in the cavity is typically air although other gases or gas mixtures are also possible such as nitrogen or argon or carbon dioxide.

As the gas filled cavity has a locally lower density than the remainder of the pharmaceutical dosage form according to the invention, the pharmaceutical dosage form according to the invention is characterized by an uneven and/or inhomogenous distribution of mass. Further to this uneven or inhomogeneous distribution of mass due to material vs. gas, the regions of the pharmaceutical dosage form outside the cavity, i.e. the regions that are prepared from solid material, may comprise an inhomogeneous distribution of density, e.g. a density gradient, which may extend along one of the directions of extension of the pharmaceutical dosage form.

In a preferred embodiment, the single cavity or the at least 2 cavities have a total volume which is at least 1.0 vol.-%, or at least 2.0 vol.-%, or at least 3.0 vol.-%, or at least 4.0 vol.-%, or at least 5.0 vol.-%; more preferably at least 6.0 vol.-%, or at least 7.0 vol.-%, or at least 8.0 vol.-%, or at least 9.0 vol.-%, or at least 10.0 vol.-%; still more preferably at least 11.0 vol.-%, or at least 12.0 vol.-%, or at least 13.0 vol.-%, or at least 14.0 vol.-%, or at least 15.0 vol.-%; yet more preferably at least 16.0 vol.-%, or at least 17.0 vol.-%, or at least 18.0 vol.-%, or at least 19.0 vol.-%, or at least 20.0 vol.-%; even more preferably at least 21.0 vol.-%, or at least 22.0 vol.-%, or at least 23.0 vol.-%, or at least 24.0 vol.-%, or at least 25.0 vol.-%; most preferably at least 26.0 vol.-%, or at least 27.0 vol.-%, or at least 28.0 vol.-%, or at least 29.0 vol.-%, or at least 30.0 vol.-%; and in particular at least 31.0 vol.-%, or at least 32.0 vol.-%, or at least 33.0 vol.-%, or at least 34.0 vol.-%, or at least 35.0 vol.-%, or at least 36.0 vol.-%, or at least 37.0 vol.-%, or at least 38.0 vol.-%, or at least 39.0 vol.-%, or at least 40.0 vol.-%, or at least 41.0 vol.-%, or at least 42.0 vol.-%, or at least 43.0 vol.-%, or at least 44.0 vol.-%, or at least 45.0 vol.-%, in each case of the total volume of the dosage form.

Preferably, the single cavity or the at least 2 cavities have a total volume which is at least 0.5 μl, or at least 1.0 μl, or at least 1.5 μl, or at least 2.0 μl, or at least 2.5 μl, or at least 3.0 μl, or at least 3.5 μl, or at least 4.0 μl, or at least 4.5 μl, or at least 5.0 μl; more preferably at least 5.5 μl, or at least 6.0 μl, or at least 6.5 μl, or at least 7.0 μl, or at least 7.5 μl, or at least 8.0 μl, or at least 8.5 μl, or at least 9.0 μl, or at least 9.5 μl, or at least 10 μl; still more preferably at least 11 μl, or at least 12 μl, or at least 13 μl, or at least 14 μl, or at least 15 μl, or at least 16 μl, or at least 17 μl, or at least 18 μl, or at least 19 μl, or at least 20 μl; yet more preferably at least 21 μl, or at least 22 μl, or at least 23 μl, or at least 24 μl, or at least 25 μl, or at least 26 μl, or at least 27 μl, or at least 28 μl, or at least 29 μl, or at least 30 μl; even more preferably at least 41 μl, or at least 42 μl, or at least 43 μl, or at least 44 μl, or at least 45 μl, or at least 46 μl, or at least 47 μl, or at least 48 μl, or at least 49 μl, or at least 50 μl; most preferably at least 51 μl, or at least 52 μl, or at least 53 μl, or at least 54 μl, or at least 55 μl, or at least 56 μl, or at least 57 μl, or at least 58 μl, or at least 59 μl, or at least 60 μl; and most preferably at least 61 μl, or at least 62 μl, or at least 63 μl, or at least 64 μl, or at least 65 μl, or at least 66 μl, or at least 67 μl, or at least 68 μl, or at least 69 μl, or at least 70 μl.

Preferably, the single cavity or the at least 2 cavities have a total volume which is at most 500 μl, or at most 490 μl, or at most 480 μl, or at most 470 μl, or at most 460 μl, or at most 450 μl, or at most 440 μl, or at most 440 μl, or at most 420 μl, or at most 410 μl, or at most 400 μl, or at most 390 μl, or at most 380 μl, or at most 370 μl, or at most 360 μl, or at most 350 μl, or at most 340 μl, or at most 330 μl, or at most 320 μl, or at most 310 μl, or at most 300 μl, or at most 290 μl, or at most 280 μl, or at most 270 μl, or at most 260 μl, or at most 250 μl, or at most 240 μl, or at most 230 μl, or at most 220 μl, or at most 210 μl, or at most 200 μl, or at most 190 μl, or at most 180 μl, or at most 170 μl, or at most 160 μl, or at most 150 μl, or at most 140 μl, or at most 130 μl, or at most 120 μl, or at most 110 μl, or at most 100 μl, or at most 99 μl, or at most 98 μl, or at most 97 μl, or at most 96 μl, or at most 95 μl, or at most 94 μl, or at most 93 μl, or at most 92 μl, or at most 91 μl; more preferably at most 90 μl, or at most 89 μl, or at most 88 μl, or at most 87 μl, or at most 86 μl, or at most 85 μl, or at most 84 μl, or at most 83 μl, or at most 82 μl, or at most 81 μl; still more preferably at most 80 μl, or at most 89 μl, or at most 88 μl, or at most 87 μl, or at most 86 μl, or at most 85 μl, or at most 84 μl, or at most 83 μl, or at most 82 μl, or at most 81 μl; yet more preferably at most 79 μl, or at most 78 μl, or at most 77 μl, or at most 76 μl, or at most 75 μl, or at most 74 μl, or at most 73 μl, or at most 72 μl, or at most 71 μl, or at most 70 μl; even more preferably at most 69 μl, or at most 68 μl, or at most 67 μl, or at most 66 μl, or at most 65 μl, or at most 64 μl, or at most 63 μl, or at most 62 μl, or at most 61 μl, or at most 60 μl; and most preferably at most 59 μl, or at most 58 μl, or at most 57 μl, or at most 56 μl, or at most 55 μl, or at most 54 μl, or at most 53 μl, or at most 52 μl, or at most 51 μl, or at most 50 μl, or at most 49 μl, or at most 48 μl, or at most 47 μl, or at most 46 μl, or at most 45 μl, or at most 44 μl, or at most 43 μl, or at most 42 μl, or at most 41 μl.

Preferably, the single cavity or the at least 2 cavities have a total volume which is in the range of 5.0±4.5 μl, or of 5.0±4.0 μl, or of 5.0±3.5 μl, or of 5.0±3.0 μl, or of 5.0±2.5 μl, or of 5.0±2.0 μl, or of 5.0±1.5 μl, or of 5.0±1.0 μl; more preferably in the range of 10.0±9.0 μl, or of 10.0±8.0 μl, or of 10.0±7.0 μl, or of 10.0±6.0 μl, or of 10.0±5.0 μl, or of 10.0±4.0 μl, or of 10.0±3.0 μl, or of 10.0±2.0 μl, or of 10.0±1.0 μl; or of 20.0±16.0 μl, or of 20.0±14.0 μl, or of 20.0±12.0 μl, or of 20.0±10.0 μl, or of 20.0±9.0 μl, or of 20.0±8.0 μl, or of 20.0±7.0 μl, or of 20.0±6.0 μl, or of 20.0±5.0 μl, or of 20.0±4.0 μl, or of 20.0±3.0 μl, or of 20.0±2.0 μl, or of 20.0±1.0 μl; or of 30.0±26.0 μl, or of 30.0±24.0 μl, or of 30.0±22.0 μl, or of 30.0±20.0 μl, or of 30.0±18.0 μl, or of 30.0±16.0 μl, or of 30.0±14.0 μl, or of 30.0±12.0 μl, or of 30.0±10.0 μl, or of 30.0±9.0 μl, or of 30.0±8.0 μl, or of 30.0±7.0 μl, or of 30.0±6.0 μl, or of 30.0±5.0 μl, or of 30.0±4.0 μl, or of 30.0±3.0 μl, or of 30.0±2.0 μl, or of 30.0±1.0 μl; or of 40.0±15.0 μl, or of 40.0±10.0 μl, or of 40.0±5.0 μl, or of 50.0±15.0 μl, or of 50.0±10.0 μl, or of 50.0±5.0 μl, or of 60.0±15.0 μl, or of 60.0±10.0 μl, or of 60.0±5.0 μl, or of 70.0±15.0 μl, or of 70.0±10.0 μl, or of 70.0±5.0 μl, or of 80.0±15.0 μl, or of 80.0±10.0 μl, or of 80.0±5.0 μl, or of 90.0±15.0 μl, or of 90.0±10.0 μl, or of 90.0±5.0 μl, or of 100.0±50.0 μl, or of 100.0±40.0 μl, or of 100.0±30.0 μl, or of 100.0±20.0 μl, or of 100.0±10.0 μl, or of 150.0±50.0 μl, or of 150.0±40.0 μl, or of 150.0±30.0 μl, or of 150.0±20.0 μl, or of 150.0±10.0 μl, or of 200.0±50.0 μl or of 200.0±40.0 μl, or of 200.0±30.0 μl, or of 200.0±20.0 μl, or of 200.0±10.0 μl, or of 250.0±50.0 μl, or of 250.0±40.0 μl, or of 250.0±30.0 μl, or of 250.0±20.0 μl, or of 250.0±10.0 μl, or of 300.0±50.0 μl or of 300.0±40.0 μl, or of 300.0±30.0 μl or of 300.0±20.0 μl, or of 300.0±10.0 μl, or of 350.0±50.0 μl, or of 350.0±40.0 μl, or of 350.0±30.0 μl, or of 350.0±20.0 μl, or of 350.0±10.0 μl, or of 400.0±50.0 μl or of 400.0±40.0 μl, or of 400.0±30.0 μl or of 400.0±20.0 μl, or of 400.0±10.0 μl, or of 450.0±50.0 μl, or of 450.0±40.0 μl, or of 450.0±30.0 μl, or of 450.0±20.0 μl, or of 450.0±10.0 μl, or of 500.0±50.0 μl or of 500.0±40.0 μl, or of 500.0±30.0 μl or of 500.0±20.0 μl, or of 500.0±10.0 μl, or of 550.0±50.0 μl, or of 550.0±40.0 μl, or of 550.0±30.0 μl, or of 550.0±20.0 μl, or of 550.0±10.0 μl.

In a preferred embodiment, the balance point of the pharmaceutical dosage form according to the invention is outside the geometrical center of the pharmaceutical dosage form (see FIG. 3). Thus, preferably the one or more cavities and the remainder of the pharmaceutical dosage form according to the invention are spatially arranged in a non-centro-symmetric manner. In consequence, after intake of the pharmaceutical dosage form, when it comes into contact with gastric fluid, the pharmaceutical dosage form floats and orients itself such that the balance point is downside, preferably below the surface of the gastric fluid.

In other words, according to this preferred embodiment, the pharmaceutical dosage form has a predetermined side that in the floating state under in vitro conditions in the absence of any stirring, shaking, motility and the like, is in contact with the gastric fluid, and another predetermined side that is not in contact with the gastric fluid.

For the purpose of the specification, unless expressly stated otherwise, any definition of the side or outer surface of the pharmaceutical dosage form that is in contact with gastric fluid or that is not in contact with gastric fluid under in vitro conditions refers to the state of the pharmaceutical dosage form immediately after it has been contacted with gastric fluid or artificial gastric fluid, respectively. Thus, any changes of the pharmaceutical dosage form that occur in the course of drug release are not to be taken into account.

Preferably, under these in vitro conditions, at least 1.0%, or at least 2.0%, or at least 3.0%, or at least 4.0%, or at least 5.0%; more preferably at least 6.0%, or at least 7.0%, or at least 8.0%, or at least 9.0%, or at least 10%; still more preferably at least 11%, or at least 12%, or at least 13%, or at least 14%, or at least 15%; yet more preferably at least 16%, or at least 17%, or at least 18%, or at least 19%, or at least 20%; even more preferably at least 21%, or at least 22%, or at least 23%, or at least 24%, or at least 25%; most preferably at least 26%, or at least 27%, or at least 28%, or at least 29%, or at least 30%; and in particular at least 31%, or at least 32%, or at least 33%, or at least 34%, or at least 35%; of the outer surface of the pharmaceutical dosage form according to the invention is not in contact with the gastric fluid, i.e. above the fluid surface.

According to a preferred embodiment, the pharmaceutical dosage form has a predetermined side that in the floating state under in vitro conditions in the absence of any stirring, shaking, motility and the like, is in contact with the gastric fluid and said predetermined side comprises two areas:
a drug releasing area that upon contact with gastric fluid releases the pharmacologically active ingredient into the gastric fluid; and
an enteric area that comprises or is covered with an enteric coating material that is essentially insoluble is gastric fluid under acidic conditions.

The outer surface of the pharmaceutical dosage form according to the invention in the floating state under in vitro conditions in the absence of any stirring, shaking, motility and the like is not in contact with the gastric fluid, i.e. above the fluid surface, may optionally also comprise or be covered with an enteric coating material that is essentially insoluble in gastric fluid under acidic conditions.

Suitable enteric coating materials are commercially available (e.g. under the tradename Eudragit®).

The size of the drug releasing area can be adjusted to the desired in vitro release profile that is to be achieved by the pharmaceutical dosage form according to the invention. In general, the larger the drug releasing area the faster the release of the pharmacologically active ingredient. Providing the pharmaceutical dosage form according to the invention with a drug releasing area and with an enteric area is advantageous with respect to a reproducible, adjustable and controlled drug release. Preferably, the drug releasing area comprises an erodible matrix material in which the pharmacologically active ingredient is embedded.

Preferably, the drug releasing area amounts to at most 99%, or at most 98%, or at most 97%, or at most 96%, or at most 95%; more preferably at most 94%, or at most 93%, or at most 92%, or at most 91%, or at most 90%; still more preferably at most 89%, or at most 88%, or at most 87%, or at most 86%, or at most 85%; yet more preferably at most 84%, or at most 83%, or at most 82%, or at most 81%, or at most 80%; even more preferably at most 89%, or at most 88%, or at most 87%, or at most 86%, or at most 85%; most preferably at most 84%, or at most 83%, or at most 82%, or at most 81%, or at most 80%; and in particular at most 79%, or at most 78%, or at most 77%, or at most 76%, or at most 75%; of the outer surface of the pharmaceutical dosage form according to the invention that in the floating state under in vitro conditions in the absence of any stirring, shaking, motility and the like is in contact with the gastric fluid; or of the total outer surface of the pharmaceutical dosage form according to the invention.

According to a preferred embodiment (see FIG. 4), the pharmaceutical dosage form according to the invention comprises
a floating unit preferably comprising the one or more cavities; and
a releasing unit comprising the pharmacologically active ingredient; and
optionally, a protecting unit.

Preferably, the floating unit comprises a material which surrounds the one or more cavities and which is insoluble or poorly soluble in gastric fluid (e.g. an enteric material). In other words, the one or more cavities are preferably embedded in the floating unit. Preferably, the floating unit comprises or is covered with an enteric coating material that is essentially insoluble in gastric fluid under acidic conditions. The purpose of the floating unit preferably is to ensure buoyancy of the pharmaceutical dosage form in gastric fluid for an extended period of time. Thus, the floating unit and hence also the one or more cavities preferably remain intact for an extended period of time during which the releasing unit may release the pharmacologically active ingredient in a floating state of the pharmaceutical dosage form. Preferably, the floating unit is composed of a material that can be three-dimensionally printed, preferably by fused deposition modeling.

Preferably, the releasing unit comprises an erodible matrix material in which the pharmacologically active ingredient is embedded. Suitable erodible matrix materials are known to the skilled person and besides the pharmacologically active ingredient may comprise a mixture of excipients such as fillers, binders, disintegrants, and the like. Preferably, the releasing unit is composed of a material that can be three-dimensionally printed, preferably by fused deposition modeling.

The total outer volume of the pharmaceutical dosage form encompasses the floating unit, the releasing unit and the optionally present protecting unit. Once the optionally present protecting unit has been dissolved, the remainder comprising the floating unit and the releasing unit preferably has a density below the density of gastric fluid such that said remainder is capable of floating.

The shape of the releasing unit of the pharmaceutical dosage form is not particularly limited. Suitable shapes include but are not limited to round, cylindrical, cone shaped, oval, quadrangular or polygonal.

In a preferred embodiment, the releasing unit comprises subunits that are preferably connected to one another and that differ in size, shape and/or composition, preferably in order
to achieve a different release kinetics of the pharmacologically active ingredient from each subunit (e.g. due to a larger and smaller surface, respectively), and/or
to influence the floating properties of the pharmaceutical dosage form, e.g. to keep and maintain balance during flotation; and/or
to optimize the available space within the pharmaceutical dosage form.

The floating unit and the releasing unit are preferably linked to one another such that the buoyancy of the floating unit prevents the releasing unit from sinking, whereas the releasing unit preferably is in contact with the gastric fluid as to release the pharmacologically active ingredient into the gastric fluid according to the desired release kinetics.

Preferably, the optionally present protecting unit comprises a material which covers at least a portion of the outer surface of the floating unit and/or at least a portion of the outer surface of the releasing unit and which is preferably soluble in gastric fluid. The purpose of the optionally present protecting unit preferably is to provide the pharmaceutical dosage form with an outer shape that can be easily swallowed by a patient without being harmful, e.g. due to sharp edges, and to liberate the floating unit and the releasing unit after intake of the pharmaceutical dosage form. Preferably, the optionally present protecting unit is dissolved within minutes after intake of the pharmaceutical dosage form thereby liberating the floating unit and the releasing unit. Preferably, the optionally present protecting unit is composed of a material that can be three-dimensionally printed, preferably by fused deposition modeling.

According to a preferred embodiment, the pharmaceutical dosage form according to the invention comprises one or more cavities that remain intact for an extended period of time during which the releasing unit may release the pharmacologically active ingredient in a floating state of the pharmaceutical dosage form as well as one or more pockets that may also be filled with gas and initially also serve as cavity. The difference of the one or more cavities on the one hand and the one or more pockets on the other hand is that the pockets preferably do not remain intact for an extended period of time during which the releasing unit may release the pharmacologically active ingredient in a floating state of the pharmaceutical dosage form (see FIG. 2).

According to a preferred embodiment, the pockets have openings at the outer surface of the pharmaceutical dosage form such that upon contact with gastric fluid, said fluid may penetrate the pockets and thus reach interior areas of the pharmaceutical dosage form.

According to another preferred embodiment, the pockets do not have openings at the outer surface of the pharmaceutical dosage form, whereas such openings are blocked with an erodible material. Thus, according to this preferred embodiment, the pockets are initially closed and thus likewise the one or more cavities contribute to the buoyance of the pharmaceutical dosage form. Upon contact with gastric fluid, in the course of erosion and release of the pharmacologically active ingredient, however, the openings are set free such that subsequently the gastric fluid may penetrate the pockets and thus reach interior areas of the pharmaceutical dosage form.

In either embodiment the pockets contribute to the overall release kinetics of the pharmacologically active ingredient from the pharmaceutical dosage form, as they shorten diffusion and erosion pathways of the gastric fluid. On the other hand, buoyancy of the pharmaceutical dosage form is ensured by the one or more cavities that remain intact while the one or more cavities may be flooded with gastric fluid.

According to a preferred embodiment, the pharmaceutical dosage form according to the invention provides prolonged release of the pharmacologically active ingredient.

Preferably, under in vitro conditions in 900 mL artificial (simulated) gastric fluid (pH 1.2 HCl) in accordance with Ph. Eur. paddle method, at 50 rpm and 37° C., the pharmaceutical dosage form according to the invention exhibits a release profile, wherein the pharmaceutical dosage form after 30 minutes has released not more than 80 wt.-%, or not more than 75 wt.-%, or not more than 70 wt.-%, or not more than 65 wt.-%, or not more than 60 wt.-%, or not more than 55 wt.-%, or not more than 50 wt.-%, or not more than 45 wt.-%, or not more than 40 wt.-%, or not more than 35 wt.-%, or not more than 30 wt.-%, or not more than 25 wt.-%, or not more than 20 wt.-%, of the pharmacologically active ingredient that was originally contained in the pharmaceutical dosage form.

Preferably, under in vitro conditions in 900 mL artificial (simulated) gastric fluid (pH 1.2 HCl) in accordance with Ph. Eur. paddle method, at 50 rpm and 37° C., the pharmaceutical dosage form according to the invention exhibits a release profile according to any of embodiments A¹ to A⁸ as compiled in the table here below:

| | | A¹ | A² | A³ | A⁴ | A⁵ | A⁶ | A⁷ | A⁸ |
|---|---|---|---|---|---|---|---|---|---|
| 30 | min | ≥5% | ≥5% | ≥5% | ≥5% | ≥5% | ≥5% | ≥5% | ≥5% |
| 60 | min | ≥10% | ≥10% | ≥10% | ≥10% | ≥10% | ≥10% | ≥10% | ≥10% |
| 2 | h | 15-70% | 20-65% | 25-60% | 30-55% | 15-60% | 20-55% | 25-50% | 30-45% |
| 4 | h | ≤75 | ≤70 | ≤65 | ≤60 | 20-65% | 25-50% | 30-45% | 35-40% |
| 6 | h | ≤80% | ≤80% | ≤80% | ≤80% | 25-70% | 30-65% | 35-60% | 40-55% |
| 9 | h | ≥80% | ≥80% | ≥80% | ≥80% | ≤75 | ≤70 | ≤65 | ≤60 |
| 12 | h | ≥95% | ≥95% | ≥95% | ≥95% | ≤80% | ≤80% | ≤80% | ≤80% |
| 18 | h | ≥95% | ≥95% | ≥95% | ≥95% | ≥80% | ≥80% | ≥80% | ≥80% |
| 24 | h | ≥95% | ≥95% | ≥95% | ≥95% | ≥95% | ≥95% | ≥95% | ≥95% |

When the pharmaceutical dosage form contains more than a single of the pharmacologically active ingredient, the above in vitro release profiles are realized by at least one of the pharmacologically active ingredients.

Preferably, the pharmaceutical dosage form according to the invention is gastric-retentive; and/or remains in floating state or buoyant for more than 1 hour, or at least 1.5 hours, or at least 2 hours, or at least 2.5 hours, or at least 3 hours, or for at least 3.5 hours, or for at least 4 hours, or for at least 4.5 hours, or for at least 5 hours, or for at least 5.5 hours, or for at least 6 hours, or for at least 6.5 hours, or for at least 7 hours, or for at least 7.5 hours, or for at least 8 hours, or for at least 8.5 hours, or for at least 9 hours, or for at least 9.5 hours, or for at least 10 hours, or for at least 10.5 hours, or for at least 11 hours, or for at least 11.5 hours, or for at least 12 hours, or after oral intake or under simulated conditions, i.e. under in vitro conditions in 900 mL artificial (simulated) gastric fluid (pH 1.2 HCl) in accordance with Ph. Eur. paddle method, at 50 rpm and 37° C.

In preferred embodiments B¹ to B¹⁰ according to the invention, the dosage form under in vitro conditions in 900 mL artificial (simulated) gastric fluid (pH 1.2 HCl) in accordance with Ph. Eur. paddle method, at 50 rpm and 37° C.,
remains in floating state for at least X hours,
after Y hours has released not more than 50 wt.-%, preferably not more than 40 wt.-%, and
after Z hours has released at least 80 wt.-%, preferably at least 90 wt.-%,
   of the pharmacologically active ingredient that was originally contained in the pharmaceutical dosage form, wherein X, Y and Z have one of the following meanings:

| | B¹ | B² | B³ | B⁴ | B⁵ | B⁶ | B⁷ | B⁸ | B⁹ | B¹⁰ |
|---|---|---|---|---|---|---|---|---|---|---|
| X | 3 | 4 | 5 | 6 | 7 | 8 | 9 | 10 | 11 | 12 |
| Y | 1 | 1.3 | 1.7 | 2 | 2.3 | 2.7 | 3 | 3.3 | 3.7 | 4 |
| Z | 3 | 4 | 5 | 6 | 7 | 8 | 9 | 10 | 11 | 12 |

To maintain buoyancy during drug release, it is important that the one or more cavities remain intact for a sufficient period of time. This may be achieved by different means.

Preferably, the cavity is encased by a material. Thus, the part of the material adjacent to the cavity, i.e. in contact with the gas within the cavity, may be regarded as a wall of the cavity (casing, cladding).

In a preferred embodiment, the cavity is encased by an enteric material, i.e. said wall is composed of or covered with an enteric material such that the gastric fluid may not dissolve the cavity wall (see FIG. 7). In a preferred embodiment, the wall is layered, wherein an enteric material forms one of the layers. For example, the enteric material may cover the outer surface of the wall, i.e. away from the cavity (see FIG. 8), or the inner surface of the wall, i.e. towards the cavity.

In a preferred embodiment, the enteric material is located towards the inside of the cavity, i.e. faces the cavity (see FIG. 7). In another preferred embodiment, the enteric material is located towards the outside of the cavity (see FIG. 8). In consequence, in either case, the enteric material forms a barrier against the gastric fluid and prevents entry of the gastric fluid into the cavity, at least for some time during which the cavity serves as floating unit and provides buoyancy to the pharmaceutical dosage form.

In a preferred embodiment, the dosage form comprises
a floating unit comprising the cavity and an enteric material, wherein the cavity is encased by the enteric material; and
a releasing unit comprising the pharmacologically active ingredient,
   wherein the floating unit is surrounded on all sides by the releasing unit, i.e. the pharmacologically active ingredient is not encased by the enteric material but exposed to gastric fluid and subject to erosion and dissolution. In consequence, the buoyancy provided by the cavity prevents the dosage form from sinking, whereas the release unit is in contact with the gastric fluid as to release the pharmacologically active ingredient into the gastric fluid according to the desired release kinetics.

In a preferred embodiment, the dosage form comprises
a floating unit comprising more than one cavity and an enteric material, wherein at least one cavity is encased by the enteric material or at least two cavities, more preferred at least three cavities, even more preferred at least four cavities are encased by the enteric material; and
a releasing unit comprising the pharmacologically active ingredient,
   wherein the floating unit is surrounded on all sides by the releasing unit, i.e. the pharmacologically active ingredient is not encased by the enteric material but exposed to gastric fluid and subject to erosion and dissolution. In consequence, the buoyancy provided by the at least one cavity or the at least two, three or four cavities prevents the dosage form from sinking, whereas the release unit is in contact with the gastric fluid as to release the pharmacologically active ingredient into the gastric fluid according to the desired release kinetics.

In a preferred embodiment, the dosage form comprises
a floating unit comprising a single cavity and an enteric material, wherein said single cavity is encased by the enteric material; and
a releasing unit comprising the pharmacologically active ingredient,
   wherein the floating unit is surrounded on all sides by the releasing unit, i.e. the pharmacologically active ingredient is not encased by the enteric material but exposed to gastric fluid and subject to erosion and dissolution. In consequence, the buoyancy provided by the single cavity prevents the dosage form from sinking, whereas the release unit is in contact with the gastric fluid as to release the pharmacologically active ingredient into the gastric fluid according to the desired release kinetics.

Preferably, said enteric material is essentially insoluble in gastric fluid under acidic conditions. Preferably, entry of the gastric fluid into the cavity is prevented for at least 3 hours, or for at least 3.5 hours, or for at least 4 hours, or for at least 4.5 hours, or for at least 5 hours, or for at least 5.5 hours, or for at least 6 hours, or for at least 6.5 hours, or for at least 7 hours, or for at least 7.5 hours, or for at least 8 hours, or for at least 8.5 hours, or for at least 9 hours, or for at least 9.5 hours, or for at least 10 hours, or for at least 10.5 hours, or for at least 11 hours, or for at least 11.5 hours, or for at least 12 hours, or after oral intake.

In a preferred embodiment, the outer surface dosage form is not coated. Preferably, the outer surface of the dosage form is not coated with an enteric material.

In another preferred embodiment, the wall of the cavity is composed of an erodible material but the thickness of said wall of the cavity, which in the buoyant state is subjected to the gastric fluid, is greater than the thickness of the releasing unit. Assuming even erosion of the material, the entire releasing unit will be dissolved, i.e. the entire content of the pharmacologically active ingredient will be released into the gastric fluid, before the wall of the cavity—due to its greater thickness—will disappear thereby causing the pharmaceutical dosage form to sink (see FIG. 5). According to this embodiment, the pharmaceutical dosage form may preferably be manufactured from two pharmaceutical compositions that differ from one another preferably only in that one composition additionally contains the pharmacologically active ingredient. Said one composition is then employed for preparing the releasing unit whereas the other composition is employed for preparing the remainder of the pharmaceutical dosage form.

The time available for solubilization of the pharmacologically active ingredient is dependent on buoyancy of the dosage form in the gastric fluid and buoyancy is inter alia dependent on the volume of the cavities. Thus, the time available for solubilization of the pharmacologically active ingredient of the cavities may be controlled by adjustment of the volume of the cavity or of several cavities. Further, buoyancy ends the moment gastric fluid enters the cavity. Hence, another way to control the solubilization time is to prevent gastric fluid from entering the cavity to achieve a longer solubilization time or to encourage dissolution of the body surrounding the at least one cavity to achieve a shorter solubilization time.

Preferred embodiments of the inventive pharmaceutical dosage form comprise at least two different pharmacologically active ingredients.

In a preferred embodiment, the pharmaceutical dosage form according to the invention comprises a first segment comprising a first cavity and a second segment comprising a second cavity. Preferably, the pharmaceutical dosage form may comprise additional segments each comprising an additional cavity. Preferably, upon contact with gastric fluid, the pharmaceutical dosage form sets free the at least two segments such that the at least two segments become independent elements that are separate from one another.

In a preferred embodiment, the first segment and the second segment are substantially identical. In another preferred embodiment, the first segment and the second segment differ from one another.

In a preferred embodiment, the first segment and the second segment have different volumes. In another preferred embodiment, the first segment and the second segment have essentially the same volume.

In a preferred embodiment, the first segment and the second segment have different weights. In another preferred embodiment, the first segment and the second segment have essentially the same weight.

In a preferred embodiment, the first segment comprises a first pharmacologically active ingredient and the second segment comprises a second pharmacologically active ingredient, wherein the first pharmacologically active ingredient differs from the second pharmacologically active ingredient.

In a preferred embodiment, the first cavity and the second cavity have different volumes. In another preferred embodiment, the first cavity and the second cavity have essentially the same volume.

In a preferred embodiment, the dosage form comprises
a first segment comprising a first floating unit comprising a first cavity and an enteric material, and
a second segment comprising a second floating unit comprising a second cavity and an enteric material,
   wherein said first cavity and/or said second cavity is/are encased by the enteric material; and
a releasing unit comprising the pharmacologically active ingredient,
   wherein the first floating unit and/or the second floating unit is/are surrounded on all sides by the releasing unit, i.e. the pharmacologically active ingredient is not encased by the enteric material but exposed to gastric fluid and subject to erosion and dissolution. In consequence, the buoyancy provided by the first and/or the second cavity prevents the dosage form from sinking, whereas the release unit is in contact with the gastric fluid as to release the pharmacologically active ingredient into the gastric fluid according to the desired release kinetics.

In a preferred embodiment, only the first cavity but not the second cavity is encased by an enteric material. In consequence, after intake of such dosage form, the second cavity is exposed to gastric fluid and thus disappears faster compared to the first segment. Accordingly, once the cavity has been filled with gastric fluid, the floating state of the second segment is not maintained any longer such that the second segment leaves the stomach and enters the intestine. Thus, according to this preferred embodiment, the second cavity preferably comprises a pharmacologically active ingredient that may be released in the intestine, whereas the first segment preferably comprises a pharmacologically active ingredient should be released in the stomach.

In a preferred embodiment, the dosage form according to the invention comprises one or more pockets, which have openings at the outer surface of the pharmaceutical dosage form, wherein the pockets are positioned between the first segment and the second segment.

Preferably, the pharmaceutical dosage form according to the invention comprises at least two segments, whereas the one or more pockets are positioned between the at least two segments. Preferably, the pockets are positioned in such a way that upon contact with the gastric fluid the dosage form is divided in at least two segments each containing at least one cavity and thus becoming independent and separate from the other segment.

Preferably, the pharmaceutical dosage form according to the invention is for use in therapy, wherein the dosage form is administered orally once daily or twice daily.

### BRIEF DESCRIPTION OF THE DRAWINGS

Preferred embodiments of the pharmaceutical dosage form according to the invention are also illustrated by FIGS. 1 to 8.

FIG. 1A schematically illustrates a cross-section of a comparatively simple embodiment of the pharmaceutical dosage form (1) according to the invention comprising body (2) which contains the pharmacologically active ingredient as well as typically one or more pharmaceutical excipients and a single cavity (3). FIG. 1B schematically illustrates a cross-section of a similar dosage form (1) according to the invention comprising two cavities (3) of about the same size. FIG. 1C schematically illustrates a cross-section of a related pharmaceutical dosage form (1) according to the invention comprising three cavities (3a,b), where cavities (3a) are of about the same size and cavity (3b) is larger.

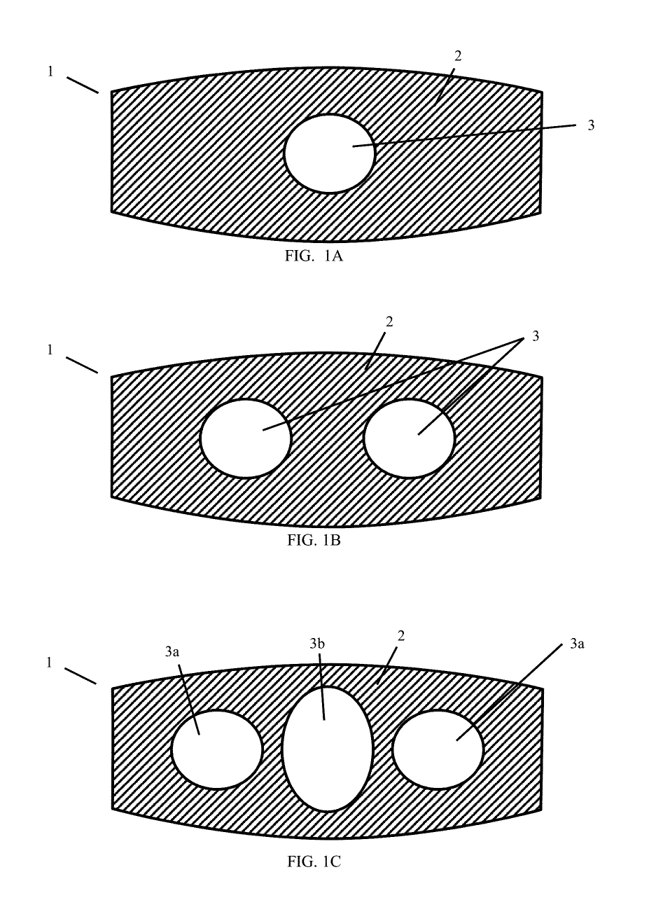

FIG. 2A schematically illustrates a cross-section of a preferred embodiment of the pharmaceutical dosage form (1) according to the invention comprising body (2) which contains the pharmacologically active ingredient as well as typically one or more pharmaceutical excipients, cavity (3) and a plurality of pockets (4) that—once their ends are exposed to gastric fluids—serve as canals allowing the gastric fluid to penetrate from the outside through the pockets into the inner core of the pharmaceutical dosage form (1). FIG. 2B schematically illustrates a cross-section of a variant of the pharmaceutical dosage form of FIG. 2B. FIG. 2C schematically illustrates a cross-section of another variant of the pharmaceutical dosage form of FIGS. 2A and 2B comprising two cavities (3) of about the same size.

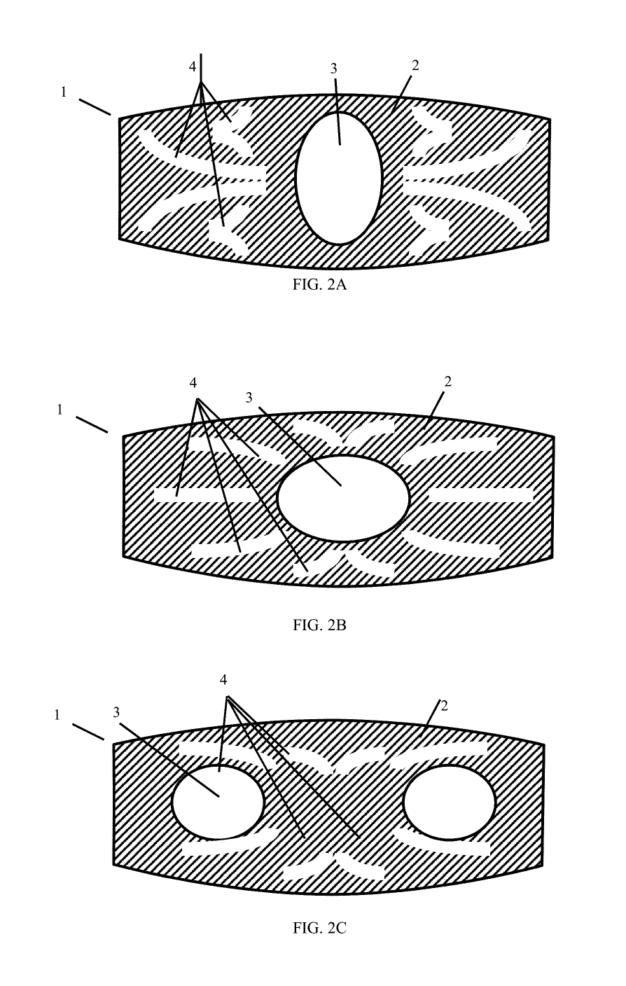

FIG. 3A schematically illustrates a cross-section of a preferred embodiment of the pharmaceutical dosage form (1) according to the invention comprising body (2) which contains the pharmacologically active ingredient as well as typically one or more pharmaceutical excipients and cavity (3), which is located above the geometrical center of the pharmaceutical dosage form (1). In consequence, the balance point of the pharmaceutical dosage form is located below its geometrical center. FIG. 3B schematically illustrates a cross-section of a variant of the pharmaceutical dosage form of FIG. 3B comprising two cavities (3) of about the same size, which are both located above the geometrical center of the pharmaceutical dosage form (1). FIG. 3C schematically illustrates a cross-section of another variant of the pharmaceutical dosage form of FIG. 2A. Cavity (3) is located on the left hand side whereas the right hand side comprises pockets (4). When being contacted with gastric fluid, the pharmaceutical dosage form will turn clockwise and float with cavity (3) upwards and pockets (4) downwards.

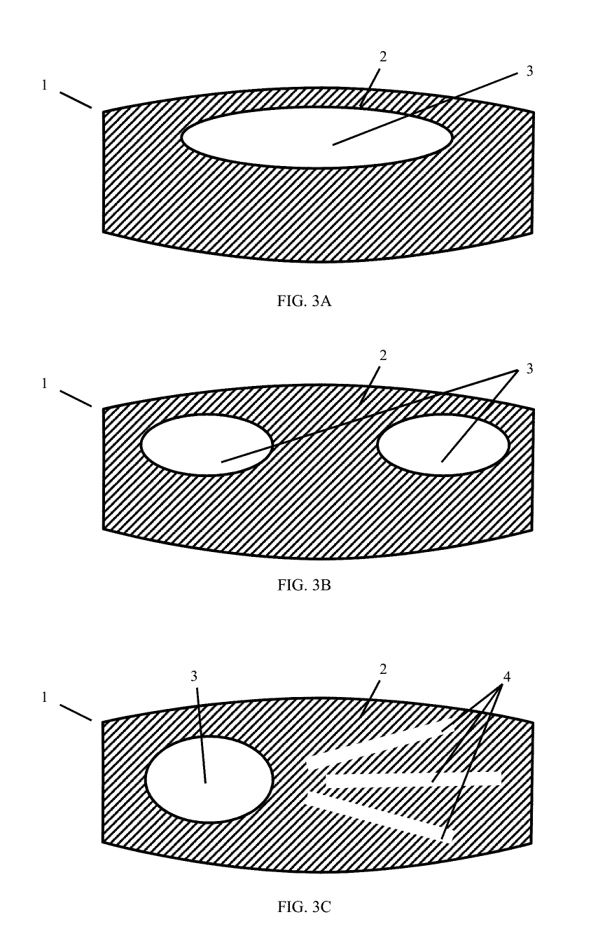

FIG. 4A/B schematically illustrate a cross-section of a preferred embodiment of the pharmaceutical dosage form (1) according to the invention. FIG. 4A schematically illustrates the state before the pharmaceutical dosage form is subjected to gastric fluid. Under these conditions, the pharmaceutical dosage form comprises body (2) which contains the pharmacologically active ingredient as well as typically one or more pharmaceutical excipients and cavity (3), which is located above the geometrical center of the pharmaceutical dosage form (1). Cavity (3) serves as floating unit whereas body (2) serves as releasing unit through its releasing area (5). Floating unit (3) and releasing unit (2) are surrounded by a protecting unit (6). FIG. 4B schematically illustrates the state after the pharmaceutical dosage form has been subjected to gastric fluid and protecting unit (6) has been dissolved thereby exposing releasing area (5) of the releasing unit (2) to the gastric fluid (7). Under these conditions, the pharmaceutical dosage form releases the pharmacologically active ingredient from the releasing unit (2) through the releasing area (5) into the gastric fluid (indicated by arrows).

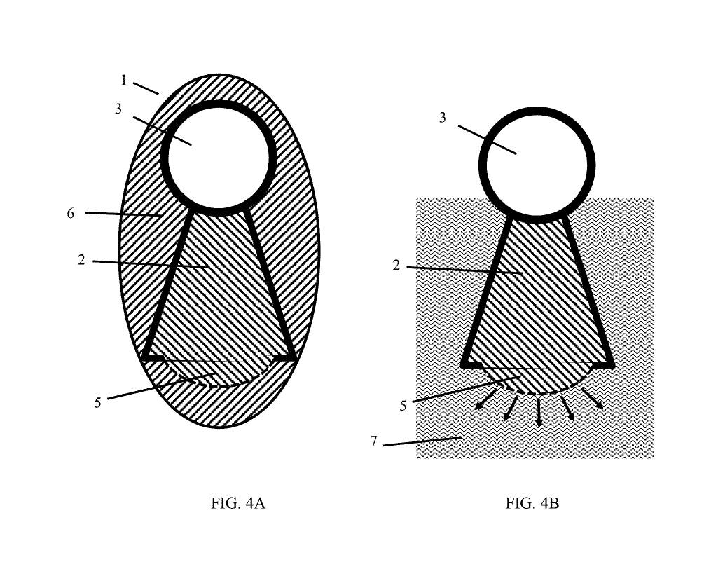

FIG. 5A/B schematically illustrate a cross-section of a preferred embodiment of the pharmaceutical dosage form (1) according to the invention. The pharmaceutical dosage form (1) schematically illustrated in FIG. 5A comprises a cavity (3), which is located above the geometrical center of the pharmaceutical dosage form (1). Cavity (3) serves as floating unit whereas body (2) serves as releasing unit through its releasing area (5). The releasing unit (2) has a thickness (b). The wall (a) of the cavity (3) is composed of an erodible material but the thickness of said wall (a) is greater than the thickness (b) of the releasing unit (2). FIG. 5B schematically illustrates a cross-section of the pharmaceutical dosage form (1) after it has been subjected to gastric fluid (7) for a while. Assuming even erosion of the material, the entire releasing unit (2) will be dissolved, i.e. the entire content of the pharmacologically active ingredient will be released into the gastric fluid (7), before the wall of the cavity (3)—due to its greater thickness (a)—will disappear thereby causing the pharmaceutical dosage form to sink.

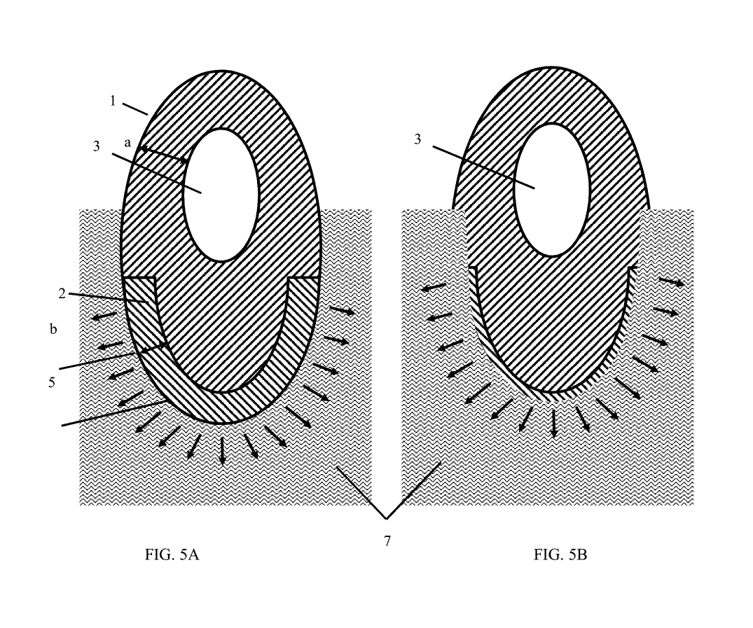

Preferably, independent from the other features depicted in FIG. 5A/B, the wall (a) of the cavity is at least 10% thicker, more preferably at least 20%, or at least 30%, or at least 40%, even more preferred at least 50%, or at least 60%, or at least 70%, or at least 80%, most preferably at least 90% thicker than the thickness (b) of the releasing unit.

FIG. 6A schematically illustrates a cross-section of an embodiment of the pharmaceutical dosage form (1) according to the invention comprising two parts (2a, 2b) of a body wherein each part contains one pharmacologically active ingredient as well as typically one or more pharmaceutical excipients. The pharmacologically active ingredient in part (2a) may differ from the pharmacologically active ingredient in part (2b). Further, each part (2a, 2b) comprises one cavity (3a, 3b), wherein the cavity (3a) in part (2a) has a bigger volume than the cavity (3b) in part (2b). The dosage form also comprises two pockets (4) that are positioned between the two parts and that—once their ends are exposed to gastric fluids—serve as canals allowing the gastric fluid to penetrate from the outside through the pockets into the inner core of the pharmaceutical dosage form (1). FIG. 6B schematically illustrates the state after the pharmaceutical dosage form has been subjected to gastric fluid and gastric fluid has penetrated from the outside through the pockets into the inner core of the pharmaceutical dosage form (1). Adjacent to the two pockets (4) the parts (2a, 2b) of the body have been partly dissolved in the gastric fluid such that the dosage form is about to be divided in two separate parts, wherein each separate part contains one cavity (3). FIG. 6C schematically illustrates the state after the body adjacent to the two pockets has been completely dissolved in the gastric fluid and the dosage form is divided in two separate parts (2a) and (2b), wherein each separate part contains one cavity (3a, 3b). Due to the bigger volume of cavity (3a) the separate part (2a) comprising this cavity will have less contact to the gastric fluid and therefore will dissolve more slowly than separate part (2b) which comprises cavity (3b) with the smaller volume and which will have more contact to the gastric fluid than separate part (2a).

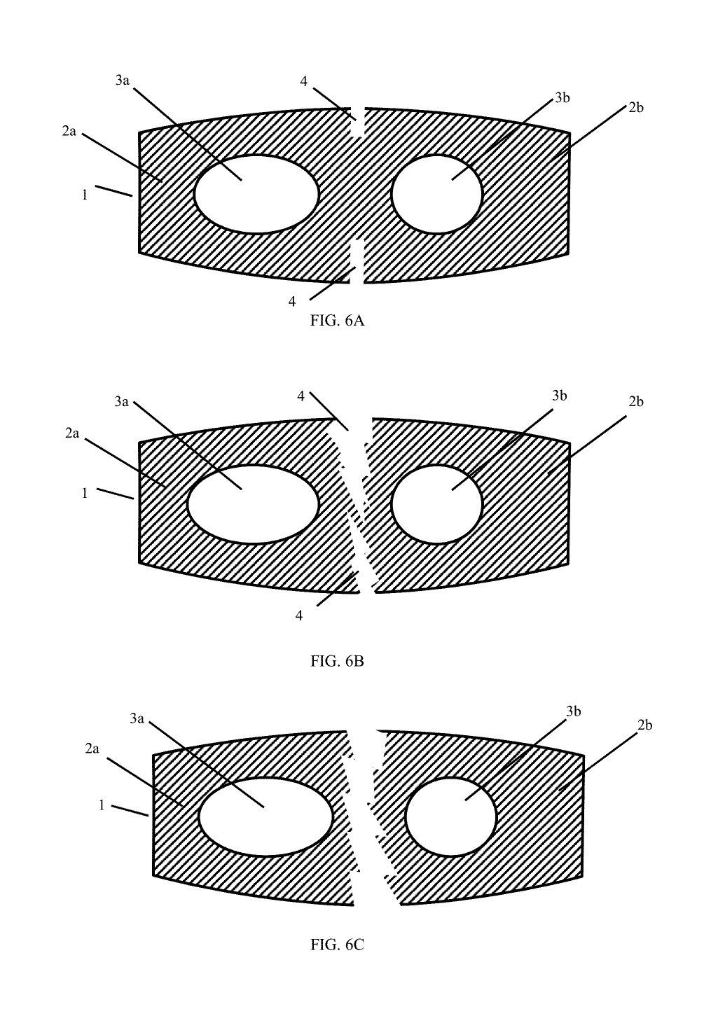

FIG. 7 schematically illustrates a cross-section of a preferred embodiment of the pharmaceutical dosage form (1) according to the invention. The pharmaceutical dosage form (1) comprises a cavity (3), which serves as floating unit, whereas body (2) serves as releasing unit. The cavity (3) is encased by an enteric material (8) on all sides by body (2), i.e. the releasing unit, i.e. the pharmacologically active ingredient is not encased by the enteric material but exposed to gastric fluid and subject to erosion and dissolution. In consequence, the buoyancy provided by the cavity prevents the dosage form from sinking, whereas the release unit is in contact with the gastric fluid as to release the pharmacologically active ingredient into the gastric fluid according to the desired release kinetics.

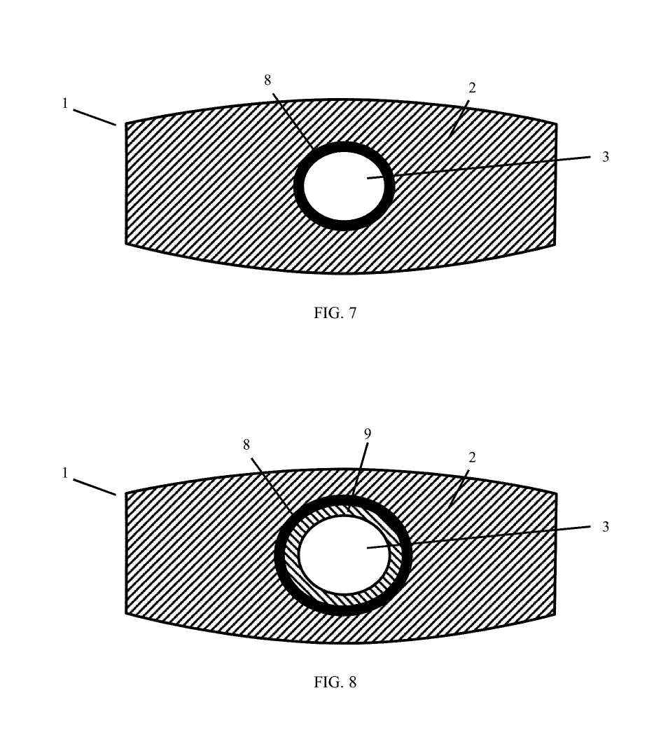

FIG. 8 schematically illustrates a variant of the pharmaceutical dosage form illustrated in FIG. 7. The pharmaceutical dosage form (1) comprises a cavity (3), which serves as floating unit whereas body (2) serves as releasing unit, whereas body (2) serves as releasing unit. According to this embodiment, the inner surface of the cavity (3) is formed by a wall (9) which in turn is covered by the enteric material (8) on its outer surface. Thus, the cavity (3) is also encased on all sides by the enteric material (8), but the enteric material (8) does not form the inner surface of the cavity (3). The pharmacologically active ingredient is not encased by the enteric material (8).

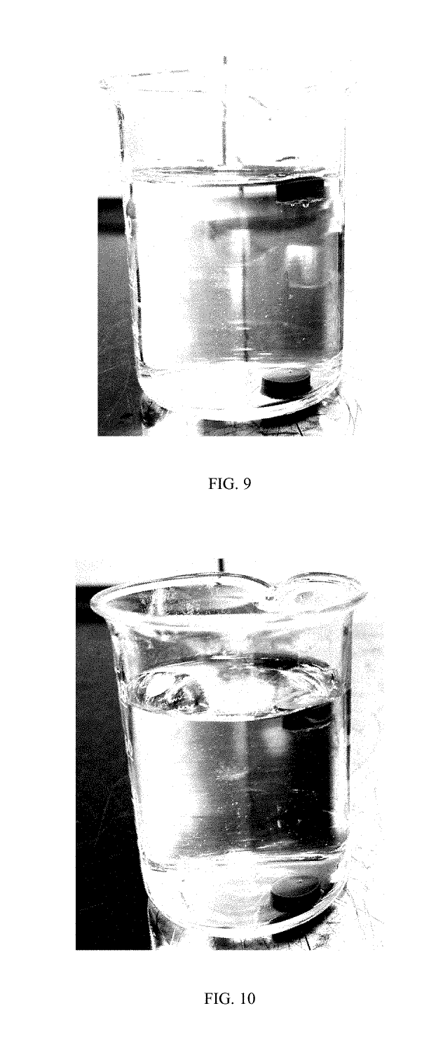

Another aspect of the invention relates to a process for the preparation of a dosage form according to the invention as described above, said process comprising a three-dimensional printing step. It has been surprisingly found that pharmaceutical dosage forms comprising comparatively large cavities can be manufactured by three-dimensional printing technologies.

Preferably, the three-dimensional printing step involves fused deposition modeling.

Machines for fused deposition modeling (FDM) are commercially available. The machines may dispense multiple materials to achieve different goals: For example, one material may be used to build up the pharmaceutical dosage form and another material may be used to build up a soluble support structure. The same applies to the materials of the floating unit, the releasing unit and the optionally present protecting unit as described above.

In FDM the pharmaceutical dosage form is produced by extruding small flattened strings of molten material to form layers as the material hardens immediately after extrusion from the nozzle. A thermoplastic filament is unwound from a coil and supplies material to an extrusion nozzle which can turn the flow on and off. A worm-drive may push the filament into the nozzle at a controlled rate. The nozzle is heated to melt the material. The thermoplastic material is heated above its glass transition temperature and is then deposited by an extrusion die. The nozzle can be moved in both horizontal and vertical directions by a numerically controlled mechanism. The nozzle follows a tool-path controlled by a computer-aided manufacturing (CAM) software package, and the pharmaceutical dosage form is built from the bottom up, one layer at a time. Stepper motors or servo motors are typically employed to move the extrusion die. The mechanism used is often an X-Y-Z rectilinear design, although other mechanical designs such as deltabot have been employed. Myriad materials are commercially available, such as polylactic acid (PLA), polyamide (PA), among many others (see Ursan et al., J Am Pharm Assoc (2003) 2013, 53(2), 136.44; Prasad et al., Drug Dev Ind Pharm 2015, 1-13).

Pharmaceutical compositions that are suitable to be employed in the three-dimensional printing step according to the invention, preferably in fused deposition modeling, are preferably identical to or at least similar with pharmaceutical compositions that have been known to be suitable for processing by conventional hot melt extrusion technology. Fused deposition modeling has many similarities with conventional hot melt extrusion.

Preferably, the pharmaceutical dosage form is prepared by three-dimensionally printing at least two different pharmaceutical compositions that preferably are provided each in form of filaments useful for fused deposition modeling. Preferably, one pharmaceutical composition contains one or more pharmacologically active ingredients, whereas the other pharmaceutical composition does not contain pharmacologically active ingredients.

Preferably, the pharmaceutical composition not containing pharmacologically active ingredients comprises or essentially consists of an enteric material. Enteric materials are known to the skilled person. Preferably, the enteric material is selected from the group consisting of methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, and mixture thereof.

Preferably, at least one cavity or the single cavity is encased by the pharmaceutical composition comprising or essentially consisting of an enteric material.

Both compositions preferably contain pharmaceutical excipients that are conventionally employed in the manufacture of pharmaceutical dosage forms, preferably in the course of three-dimensional printing technology, especially fused deposition modeling. The following preferred embodiments apply to both pharmaceutical compositions (in the following referred to as “pharmaceutical composition”), irrespective of whether they contain a pharmacologically active ingredient or not.

Preferably, the pharmaceutical composition comprises a plasticizer. Suitable plasticizers are known to the skilled person. Examples include but are not limited to polyethylene glycols, such as PEG 1500 or PEG 4000 or PEG 6000; citrates, phthalates, glycerin, sugar alcohols, various contents of copolymers (e.g. ethylene vinyl acetate (EVA)/vinyl acetate (VA)), and mixtures of any of the foregoing.

The content of plasticizer is preferably within the range of from 0.1 to 20 wt.-%, more preferably 5.0 to 17.5 wt.-%, still more preferably 7.5 to 15 wt.-%, relative to the total weight of the pharmaceutical composition.

Preferably, the pharmaceutical composition comprises one or more matrix polymers. Suitable matrix polymers are known to the skilled person. Examples include but are not limited to polylactic acid (PLA); cellulose ethers such as methylcellulose (MC), ethylcellulose (EC), hydroxypropylcellulose (HPC) and hydroxypropylmethylcellulose (HPMC); vinyl polymers such as polyvinylpyrrolidone (e.g. Kollidon® PF 12) or blends thereof such as polyvinyl acetate/polyvinylpyrrolidone (e.g. Kollidon® SR). Other suitable polymers include ethylene vinyl acetate copolymers (EVA), polyvinyl chloride, polyethylene terephthalate (PET), polyurethanes (PU), polyamides (PA), polyacrylates and mixtures of any of the foregoing. Polylactic acid (PLA) is particularly preferred.

The pharmaceutical composition may consist of one or more matrix polymers. The total content of matrix polymers is preferably within the range of from 5.0 to 95 wt.-%, more preferably 10 to 90 wt.-%, still more preferably 25 to 85 wt.-%, relative to the total weight of the pharmaceutical composition.

Representative pharmaceutical compositions that are useful for the purpose of the invention are compiled in the table here below. Composition 1 comprises a pharmacologically active ingredient whereas Composition 2 comprises the same excipients in the same absolute amounts but no pharmacologically active ingredient:

| | | Composition | | Composition | |
|---|---|---|---|---|---|
| | | 1A | | 2A | |
| | | weight | content | weight | content |
| ingredient | | [mg] | [wt.-%] | [mg] | [wt.-%] |
| pharmacologically | Tramadol | 50 | 25 | — | — |
| active ingredient | HCl | | | | |
| plasticizer | PEG 4000 | 20 | 10 | 20 | 13.3 |
| matrix polymer | Ethylcellulose | 80 | 40 | 80 | 53.3 |
| matrix polymer | Polylactic acid | 50 | 25 | 50 | 33.3 |
| | | Composition | | Composition | |
| | | 2A | | 2B | |
| | | weight | content | weight | content |
| ingredient | | [mg] | [wt.-%] | [mg] | [wt.-%] |
| pharmacologically | Tramadol | 0.5 | 0.1 | — | — |
| active ingredient | HCl | | | | |
| plasticizer | PEG 4000 | 49.5 | 9.9 | 49.5 | 9.9 |
| matrix polymer | Kollidon | 450 | 90 | 450 | 90.1 |
| | PF 12 | | | | |
| | | Composition | | Composition | |
| | | 3A | | 3B | |
| | | weight | content | weight | content |
| ingredient | | [mg] | [wt.-%] | [mg] | [wt.-%] |
| pharmacologically | Tramadol | 204.08 | 40.8 | — | — |
| active ingredient | HCl | | | | |
| matrix polymer | Kollidon SR | 255.1 | 51.0 | 255.1 | 86.2 |
| matrix polymer | HPMC | 40.81 | 8.2 | 40.81 | 13.8 |

For filament preparation, a matrix polymer or a mixture of various matrix polymers, e.g. hydroxypropylcellulose (HPC), may be stored 24 h in oven at 40° C.; when required it may be mixed in a mortar with PEG 1500 or PEG 4000 (2%, 5%, 10% by weight calculated with respect to the dry polymer). Hot-melt extrusion (HME) may be carried out in a twin-screw extruder (Haake MiniLab II, Thermo Scientific, USA) equipped with an aluminum rod-shaped die (ø 2.00 mm). Extruded rods may be calibrated and rolled up on a spool.

Another aspect of the invention relates to a pharmaceutical dosage form that is obtainable by the process according to the invention as described above.

### EXAMPLES

Two tablets were manufactured by three-dimensional printing involving fused deposition modeling. A comparative tablet was prepared from solid material, i.e. which did not comprise cavities. An inventive tablet was prepared by printing web layers above one another thereby forming cavities. Both tablets were made from the same material (polylactic acid, polylactate, PLA).

FIGS. 9 and 10 illustrate the floating behavior of the inventive tablet compared to the sinking comparative tablet.

1. A gastric-retentive pharmaceutical dosage form, which has been manufactured by three-dimensional printing technology and which has a density below the density of gastric fluid, wherein the dosage form comprises
a pharmacologically active ingredient and
a cavity, wherein the cavity has a volume of at least 0.1 μl; and
wherein the balance point of the dosage form is outside the geometrical center of the dosage form.

2. The dosage form according to claim 1, wherein the cavity has a volume of at least 25 μl.

3. The dosage form according to claim 1, comprising
a floating unit comprising the cavity; and
a releasing unit comprising the pharmacologically active ingredient; and
optionally, a protecting unit.

4. The dosage form according to claim 3, wherein the floating unit comprises a material which surrounds the cavity and which is insoluble or poorly soluble in gastric fluid.

5. The dosage form according to claim 3, wherein the releasing unit comprises an erodible matrix material in which the pharmacologically active ingredient is embedded.

6. The dosage form according to claim 3, wherein the optionally present protecting unit comprises a material which covers at least a portion of the outer surface of the floating unit and/or at least a portion of the outer surface of the releasing unit and which is soluble in gastric fluid.

7. The dosage form according to claim 1, comprising one or more pockets, which have openings at the outer surface of the pharmaceutical dosage form.

8. The dosage form according to claim 1, which comprises an enteric material.

9. The dosage form according to claim 1, comprising a first segment comprising a first cavity and a second segment comprising a second cavity.

10. The dosage form according to claim 9, wherein the first cavity and/or the second cavity is/are encased by an enteric material.

11. The dosage form according to claim 9, wherein the first cavity and the second cavity have different volumes.

12. The dosage form according to claim 9, comprising one or more pockets, which have openings at the outer surface of the pharmaceutical dosage form, wherein the pockets are positioned between the first segment and the second segment.

13. The dosage form according to claim 1, wherein the dosage form comprises a combination of two or more pharmacologically active ingredients or a combination of two or more derivatives of one and the same pharmacologically active ingredients such as different salts.

14. The dosage form according to claim 1, wherein the one or more cavities and the remainder of the pharmaceutical dosage form are spatially arranged in a non-centro-symmetric manner.

15. A process for the preparation of a dosage form according to claim 1, said process comprising a three-dimensional printing step.
